(19) United States
(12) Patent Application Publication
(10) Pub. No.: US 2023/0298754 A1
(43) Pub. Date: Sep. 21, 2023

(54) METHODS AND APPARATUS FOR DIABETES AND GLYCEMIC MANAGEMENT HAVING A DATABASE AND HANDHELD MANAGEMENT SYSTEM

(71) Applicant: Minerva Analysis LLC, Arlington, VA (US)

(72) Inventor: Janice Bitetti, Arlington, VA (US)

(73) Assignee: Minerva Analysis LLC, Arlington, VA (US)

(21) Appl. No.: 18/156,351

(22) Filed: Jan. 18, 2023

Related U.S. Application Data
(60) Provisional application No. 63/300,889, filed on Jan. 19, 2022.

(51) Int. Cl. G16H 50/20; A61B 5/145; A61B 5/0205; G16H 10/60; G16H 20/17; G16H 20/60; G16H 80/00; G16H 20/30

(52) U.S. Cl. CPC G16H 50/20; A61B 5/14532; A61B 5/02055; G16H 10/60; G16H 20/17; G16H 20/60; G16H 80/00; G16H 20/30

(57) ABSTRACT

Embodiments are directed to methods and systems for managing glucose levels in a patient including continuously collecting glucose data from the patient, processing the glucose data, and using machine learning algorithms personalized to the patient that use machine learning for continuously predicting and determining a recommended treatment for the patient. The methods and systems include a history of the glucose data and treatments is stored in a database by the patient or a caregiver. The machine learning algorithm uses the history, processed glucose data, and other personalized data to predict and calculate recommended treatment. The patient and the caregiver are provided with real-time updates of the history on demand via a web portal or mobile device.

### BACKGROUND

Glucose monitors are devices that measure the amount of glucose, or sugar, in a person's blood. These monitors are commonly used by people with diabetes to help them manage their condition and maintain healthy blood sugar levels. Glucose monitoring is typically done by pricking a finger with a small needle and placing a drop of blood on a test strip, which is then inserted into the glucose monitor for measurement. Other forms of glucose monitoring also may be used.

Glucose monitoring has traditionally been done manually, with people recording their glucose levels in a logbook and using that information to make decisions about their diabetes management. However, with the advent of technology, glucose monitors have become more advanced and integrated with mobile apps. These apps allow users to easily track and record their glucose levels, set reminders for testing, and view trends and patterns in their glucose data over time.

Some glucose monitoring apps also integrate with other devices such as continuous glucose monitors (CGMs) and insulin pumps. CGMs are devices that measure glucose levels continuously throughout the day, providing a more comprehensive view of a person's glucose levels. These devices can also send alerts to the user or their healthcare provider if glucose levels are too high or too low. Insulin pumps are devices that deliver insulin to the body.

Additionally, some glucose monitoring apps also allow for the input of other information such as food, exercise and medication intake, enabling a more holistic view of the patient health status and enabling more accurate predictions and guidance.

### SUMMARY

Methods and apparatus for management and treatment of diabetes using machine learning and data analysis to analyze individual response to carbohydrates, insulin, and food to recommend and administer therapeutic. The system may also take in exercise and sleep data entered manually, automatically, or connected with other external apps. Data from the individual's continuous glucose monitoring (CGM) and other sources is combined to create a model of individual reactions to predict how the individual's body will respond to these sources.

The present invention includes methods and apparatus for entering and recording patient data. It may include a smart device application and/or online portal for viewing historical and present patient CGM data which can be accessed via login by any device, smart watch connectivity for communication and alerts, as well as data from additional sensors such as a thermometer, exercise monitor, sleep monitor, etc. It may connect with phone-connected glucose readers, and the invention's predictive algorithm will take into account expected “cheat days” such as birthdays, holidays, and other traditional feast days which can be added into the program. If the program finds potentially harmful patterns in the patient's data, or signs of a changing condition, the patient will receive a report as well as guidance on how to improve this or what medical professional to see.

The present invention includes methods and apparatus for recommending manual therapeutic administration. This includes warning messages sent to the patient or caregiver's mobile device, personal computer, or smart watch when a glycemic event is detected, as well as recommending a suggested insulin or carbohydrate dosage calculated by the invention's algorithm. The patient can record on their device and/or smart watch when the treatment is administered, as well as their status and how they feel afterward. A feature allows the patient or caregiver to delete or correct faulty data stored in the program's databases. If the patient needs treatment at night, a feature allows the device and/or patient or caregiver's smart watch or phone or other device to vibrate to wake them.

The present invention includes methods and apparatus for using patient data to automatically administer therapeutic. This may be present at any stage of the invention's data collection or it may activate once a certain amount of data is collected and/or the machine can predict the individual's response to treatment within a certain degree of grouped in the program based on how they react to treatment, and this data analysis of similar patients is prioritized when it comes to predicting individual response. This group may have other factors in common such as age, weight, and sex. The program may prompt the individual, telling them the calculated insulin dosage and asking for approval to administer it. When the patient approves, dosage is administered and the patient and/or caregiver may be notified via an app or a text message. The program may also suggest carbohydrates for the patient to ingest. The program may also be used by a parent or guardian to make decisions about their child's insulin doses or carb boosting foods from a remote location. This may allow a figure such as a school nurse to correct the glucose levels of their child without needing to be there physically.

If the individual does not approve the app notification, there may be a protocol to gauge patient response to see if they are conscious or otherwise unwell. If the patient's glucose levels are dangerously low and they are unresponsive, possibly combined with confirmed lack of movement from a smart watch or other device with an accelerometer, the program may be permitted to inject the individual with a calculated rescue dose of glucagon and/or may be permitted to dial the appropriate emergency authority if the patient or caregiver does not respond to a set number of emergency push notifications or texts. The program may have an easily accessible stop button on the user's phone, smart watch, or insulin pump to stop treatment in case of an emergency or malfunction. Some embodiments may also include the use of a smart home device to sound an alarm or notify professionals or call a list of local people if a patient is unresponsive.

The present invention includes methods and apparatus for using patient data to predict glycemic events. Patient data is compared to that of other diabetics of their same type to look for irregularities and indicators of other health issues. Patterns in patient data are used to predict glycemic events and suggest preventative treatment in order to avoid dangerous situations, particularly in vulnerable young children.

The present invention includes methods and apparatus for facilitating communication between patient and caregiver/health care provider. Data is shareable and the application and/or program may have the capability to add users such as babysitters, notifying them of predicted glycemic events or necessary treatments. An app may facilitate communication between secondary caregivers such as babysitters and primary caregivers to ensure the patient receives the appropriate treatment. Data may be automatically uploaded to doctors, nurses, school nurses, and healthcare providers. The information may be updated in real-time so that doctors may participate in real-time treatment of patients.

Some embodiments are directed to methods and systems for managing glucose levels in a patient including continuously collecting glucose data from the patient, processing the glucose data, and using machine learning algorithms personalized to the patient that use machine learning for continuously predicting and determining a recommended treatment for the patient. The methods and systems include a history of the glucose data and treatments is stored in a database by the patient or a caregiver. The machine learning algorithm uses the history, processed glucose data, and other personalized data to predict and calculate recommended treatment. The patient and the caregiver are provided with real-time updates of the history on demand via a web portal or mobile device.

In some embodiments, the machine learning algorithm creates a model to predict how the patient will respond to various food consumptions or activities and uses machine learning to modify the model based on the processed glucose data, treatments and personalized data provided by the patient or the caregiver.

In some embodiments, the machine learning algorithm is a cloud-based machine learning algorithm that provides predictive analytics and optimization of therapeutic timing and dose delivery of insulin, naturally occurring mediators, synthetic mediators, and diet recommendations.

In some embodiments, the personalized data includes foods commonly eaten by the patient and the machine learning algorithm instructs treatment based on anticipated consumption of foods commonly eaten by the patient.

In some embodiments, the personalized data includes activities performed by the patient and the machine learning algorithm instructs future treatment based on anticipated activities of the patient.

In some embodiments, the history for the patient are accessible to the patient and the caregiver by accessing the database, and wherein the database is cloud based.

In some embodiments, personalized data is provided by the patient or the caregiver and includes wellness, food and drink consumption, and activities of the patient, and wherein faulty data included in the personalized data is removed by the patient or the caregiver.

Some embodiments include presenting instructions and educational information to the caregiver to assist the patient in critical situations.

In some embodiments, the machine learning algorithm uses machine learning to enhance accuracy of the predictions and treatments.

Some embodiments include further comprising positively rewarding the patient to encourage the patient to respond quickly to treatment instructions.

In some embodiments, the history is accessible from a mobile device with app, web portal, and smart watch.

Some embodiments include using an intervention feature to administer necessary drugs to the patient.

Some embodiments include a system for managing glucose levels in a patient including a continuous glucose monitor (CGM) configured to collect glucose data from the patient, a computer processor operatively connected to the CGM, the computer processor configured to receive and process the glucose data, a database operatively connected to the computer processor, the database storing the collected glucose data and the processed glucose data, and a decision tree algorithm being personalized to the patient and stored on the computer processor. the decision tree algorithm may be configured to predict and determine at least one recommended treatment for the patient based on the processed glucose data by using machine learning and algorithms to identify patterns based on combined collected and processed glucose data from the patient with collected and processed glucose data of one or more other patients stored in another database, and personal data of the patient provided by one of the patient, a caregiver, and a professional. The decision tree algorithm uses machine learning to make corrections or changes in real time to the recommended treatments.

Some embodiments include instructions to assist in critical situations being stored on the computer processor and is provided on demand to the patient or the caregiver. In some embodiments, the patient or caregiver provides goals and the algorithm tracks progress of the goals. In some embodiments, the algorithm determines that a goal has been met and the processor reports the met goal to the patient and the caregiver. Some embodiments include an online portal accessible by a mobile device and configured to send/receive real-time communication and alerts to the caregiver. Some embodiments include a connector for operatively connecting to a wearable thermometer, motion detector, pulse oximeter, and/or other sensors to monitor vital signs of the patient, the vital signs being at least part of the personal data. Some embodiments include a report generation feature configured to report vital signs of the patient to the caregiver on demand. In some embodiments, the decision tree algorithm uses machine learning and algorithms to identify a critical glucose data for the patient and instructs an intervention device to automatically treat the patient based on the identified critical glucose data.

### BRIEF DESCRIPTIONS OF DRAWINGS

Each figure connects sequentially as part of a larger logical process. Various exemplary aspects of the systems and methods will be described in detail, with reference to the following figures, wherein:

FIG. 1 is a flowchart depicting sensor network connectivity as well as data input and access.

FIG. 2 is a flowchart depicting the program automatically administering or recommending calculated treatment to the patient or caregiver based on sensor input and patient data.

FIG. 3 is a flowchart depicting the program using patient data to predict, prevent, and respond to glycemic events.

FIG. 4 is a flowchart depicting the program facilitating communication between the program, patient, primary caregiver, and secondary caregiver.

FIG. 5 is a flowchart depicting a possible program's decision-making tree based on the patient's glucose levels.

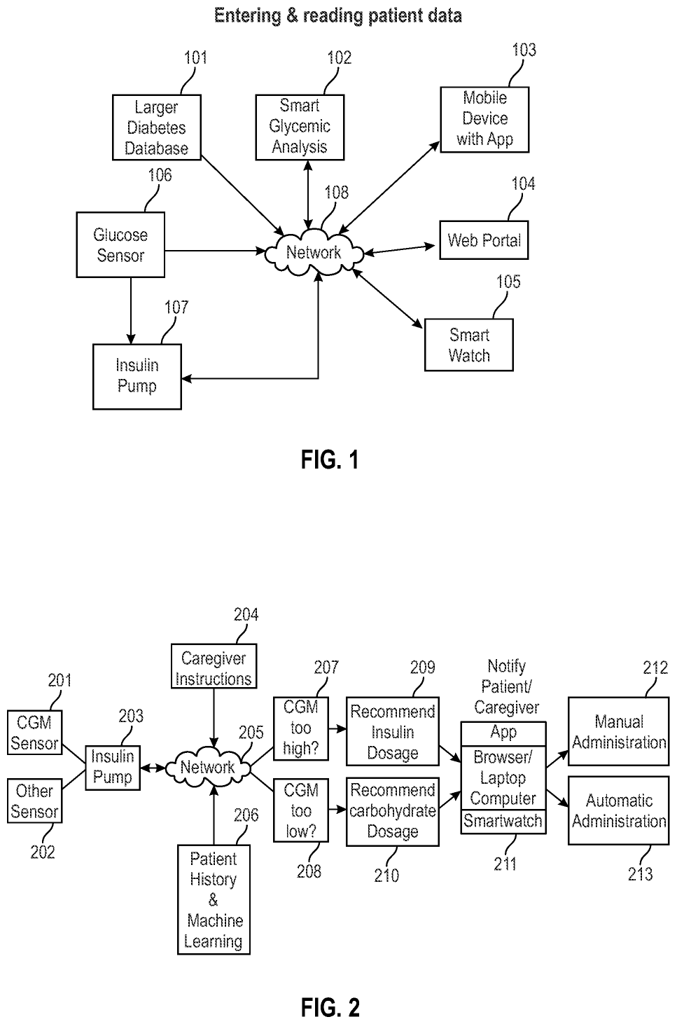

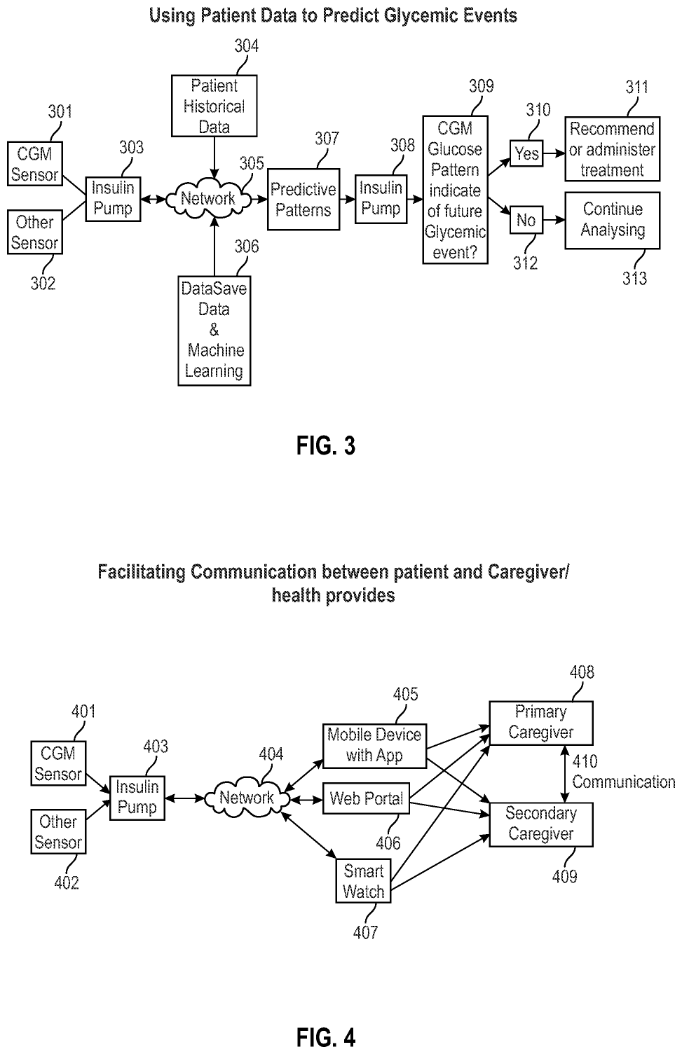

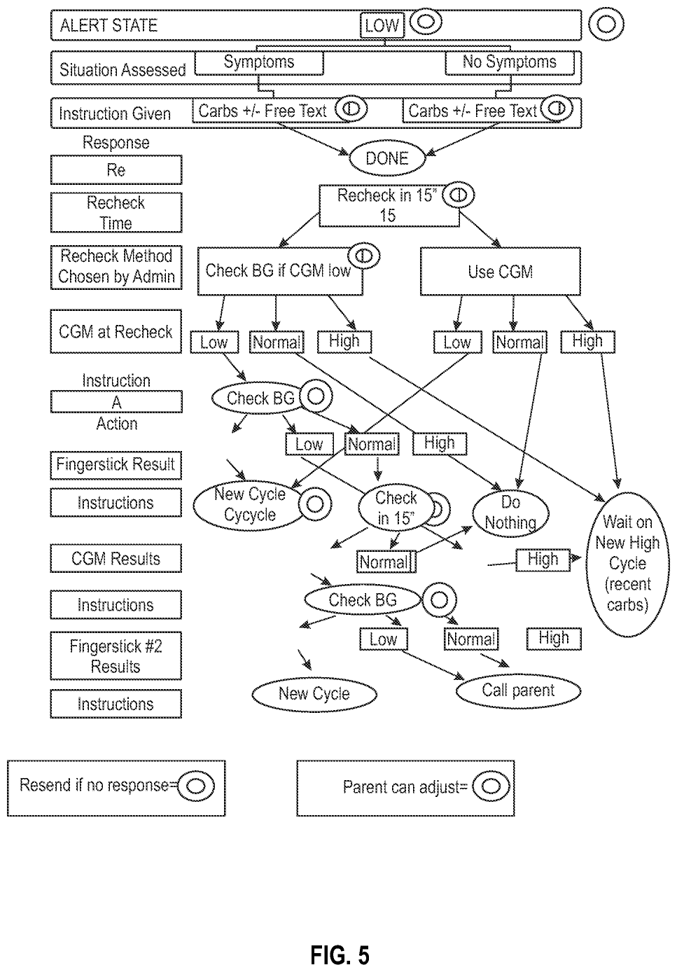

### DETAILED DESCRIPTION

These and other features and advantages are described in, or are apparent from, the following detailed description of various exemplary embodiments. Before embodiments of the invention are explained in detail, it is to be understood that the invention is not limited in its application to the details of construction and the arrangements of the components set forth in the following description or illustrated in the drawings. The invention is capable of other embodiments and of being practiced or being carried out in various ways. Also, it is understood that the phraseology and terminology used herein is for the purpose of description and should not be regarded as limiting. The use of “including” and “comprising” and variations thereof herein is meant to encompass the items listed thereafter and equivalents thereof as well as additional items. It will be understood that when an element is referred to as being “on”, “connected”,
or “coupled” to another element, it can be directly on, connected, or coupled to the other element or intervening elements that may be present. In contrast, when an element is referred to as being “directly on”, “directly connected”, or “directly coupled” to another element, there are no intervening elements present. As used herein the term “and/or” includes any and all combinations of one or more of the associated listing items. Further, it will be understood that when a layer is referred to as being “under” another layer, it can be directly under, or one or more intervening layers may also be present. In addition, it will also be understood that when a layer is referred to as being “between” two layers, it can be the only layer between the two layers, or one or more intervening layers may also be present.

It will be understood that, although the terms “first”, “second”, etc. may be used herein to describe various elements, components, regions, layers, and/or sections, these elements, components, regions, layers and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of exemplary embodiments.

In the drawing figures, the dimensions of layers and regions may be exaggerated for clarity of illustration. Like reference numerals refer to like elements throughout. The same reference numbers indicate the same components throughout the specification.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For exemplary, if the device in the figures is turned over, elements described as “below”, or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of exemplary embodiments. As used herein, the singular forms “a”, “an”, and “the” are intended to include the plural forms as well, unless the context clearly indicates otherwise. It will be further understood that the terms “comprises” and/or “comprising”, when used in this the presence of stated features, integers, steps, operations, elements, and/or components, the presence or addition of one or more other features, integers, steps, operations, elements, components, and/or groups thereof.

Exemplary embodiments are described herein with reference to cross-sectional illustrations that are schematic illustrations of idealized embodiments (and intermediate structures) of exemplary embodiments. As such, variations from the shapes of the illustrations as a result, for exemplary, of manufacturing techniques and/or tolerances, are to be expected. Thus, exemplary embodiments should not be construed as limited to the particular shapes of regions illustrated herein but are to include deviations in shapes that result, for exemplary, from manufacturing. For exemplary, an implanted region illustrated as a rectangle will, typically, have rounded or curved features and/or a gradient of implant concentration at its edges rather than a binary change from implanted to non-implanted region. Likewise, a buried region formed by the implantation may result in some implantation in the region between the buried region and the surface through which the implantation takes place. Thus, the regions illustrated in the figures are schematic in nature and their shapes are not intended to illustrate the actual shape of a region of a device and are not intended to limit to scope of exemplary embodiments.

Unless otherwise defined, all terms (including technical and scientific terms) used herein have the same meaning as commonly understood by one of ordinary skill in the art to which exemplary embodiments belong. It will be further understood that all terms, such as those defined in commonly used dictionaries, should be interpreted as having a meaning that is consistent with their meaning in the context of the relevant art and will not be interpreted in an idealized or overly formal sense unless expressly so defined herein. As used herein, expressions such as “at least one of′, when preceding a list of elements, modify the entire list of elements and do not modify the individual elements of the list.

When the terms “geometric shape is not required but that latitude for the shape is within the scope of the disclosure. Although the tubular elements of the embodiments may be cylindrical, other tubular cross-sectional forms are contemplated, such as square, rectangular, oval, triangular, and others.

Although corresponding plan views and/or perspective views of some cross-sectional view(s) may not be shown, the cross-sectional view(s) of device structures illustrated herein provide support for a plurality of device structures that extend along two different directions as would be illustrated in a plan view, and/or in three different directions as would be illustrated in a perspective view. The two different directions may or may not be orthogonal to each other. The three different directions may include a third direction that may be orthogonal to the two different directions. The plurality of device structures may be integrated in a same electronic device. For exemplary, when a device structure (e.g., a memory cell structure or transistor structure) is illustrated in a cross-sectional view, an electronic device may include a plurality of the device structures (e.g., memory cell structures or transistor structures), as would be illustrated by a plan view of the electronic device. The plurality of device structures may be arranged in an array and/or in a two-dimensional pattern.

Reference will now be made in detail to embodiments, exemplaries of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain exemplary embodiments of the present description, which is divided into the following sections.

The basic Diamon system is a personalized CGM-triggered decision tree algorithm. The idea is based on paper handouts that parents often give the school nurse or the babysitter with a list, for example: “If glucose <60, please give a juice box” and “If glucose >250 please give an insulin correction.” The list can be easily misplaced and there is no transparency: the parent might see the glucose value “60 mg/dL” on the CGM Follow app and have no idea of whether the nurse or the child saw it or did anything about it. What follows is a barrage of texts or phone calls. Diamon replaces this inadequate system. Diamon also offers another very important safety feature, the “Help Menu” with backup and educational information to assist in critical situations or even to give the babysitter a basic review of T1D. In some embodiments, Diamon offers a transparent platform so many people see the same information at the same time. Diamon can also be adjusted so that the instructions come from a health care provider versus just a parent or guardian.

Diamon consists of a decision tree algorithm for every glucose range: parents can decide upon glucose range values (for some parents “High” might be 160 mg/dL, for others it might be 140 mg/dL); for each range there are situations (e.g., symptoms, no symptoms); for each situation the parent chooses an instruction (e.g., juice, tabs, whatever) as well as a recheck time and method such as “Check fingerstick in 15” after taking carbs to treat a low.” Parents can choose to enter different sets of instructions for different situations such as overnight or soccer practice. In addition, they can enter emergency contact information. All parental instructions are stored in a MySQL Cloud database. Machine learning algorithms may also be used to predict and calculate necessary treatments. A positive reward system, discussed later, may also be implemented to encourage users, particularly children and teenagers, to respond quickly to the program's instructions. Machine learning may be used to hone those rewards to improve glucose control.

### Entering and Recording Patient Data

Some embodiments may collect blood glucose data via continuous glucose monitoring, and stores this data either in a pump device or on the web or in the cloud. Some prior art implements programs that find patterns, and this data can sometimes be accessed by caregivers or healthcare providers on the web. The data collected can sometimes be analyzed and used to recommend dosage and therapeutic treatments, as well as alert the patient when their blood glucose behaves irregularly or falls outside their recommended range. In some embodiments, this data analysis may be enhanced by using machine learning and AI in addition to standard clinical method.

The present invention connects the patient's continuous glucose monitoring (CGM) sensor 106 to the network 108, which connects to their insulin pump 107. The network-connected smart glycemic analysis 102 is able to use machine learning and algorithms to combine the patient's CGM data with that of other users of a larger diabetes database 101 and the patient's own prior data to find patterns in the patient's data. The patient's data is accessible from a mobile device with app 103, web portal 104, and smart watch 105. These three technologies can input directions and data into the program, which may have both a Cloud component and a local component, to make corrections or to customize the patient's treatment. The patient may also be able to use one or more of those three methods to input information such as carbohydrate intake, manual insulin dosage, and other outside factors such as exercise. Smart watch connectivity may be particularly important in older patients who are capable to use it to conveniently update this information and view their glucose data. It may be possible to automatically upload eating, sleep, pulse, temperature, and exercise data. In some embodiments, other sensors may be connected such as pulse, temperature, exercise patterns, food, and sleep monitors in place or in addition to the insulin monitor.

One of the benefits of being able to access patient data from a web portal 104 and mobile device 103 is that this allows caregivers to have real-time information on their patient even when not with them physically. This is one of the primary advantages of the present invention, in addition to the real-time connectivity of the many players involved in the care of someone with Type 1 or Type 2 Diabetes.

Some embodiments may include an online portal available to be accessed by any computer or smart device with a code and/or password, or smart watch compatibility for communication and alerts. Some embodiments may allow for the program to issue the patient and/or caregiver a report if there are harmful patterns in the patient's data, and this report can be shared with their healthcare provider and/or recommend what the patient can do to improve this data. Other embodiments may allow for connectivity to a wearable thermometer, motion detector, pulse oximeter, and/or other sensors, possibly included in the patient's smart watch, because low body temperatures can be indicative of hypoglycemia or other health issues and high body temperatures can indicate illness which may require treatment to be adjusted. Other embodiments may allow for connection to outside, possibly phone-connected glucose readers in addition to the ability to connect to real-time CGM. The algorithm may automatically consider or allow for the patient to manually input “cheat-days” such as birthdays, holidays, events (social activities), or traditional feast days and adjusts recommendations accordingly. Other embodiments may allow the patient and/or caregiver to enter information via smart watch, which is convenient in restaurants.

The present invention may use one or more databases, one with longitudinal data from 1 individual for close to 8 years. That data has been used to set up a system of algorithms and analytics that can be personalized for any other individual. The data includes:
Date and Time stamps;
CGM;
Finger stick glucose values;
Pump basal rates; Pump boluses; Type of bolus;
Pump carbohydrate entries;
Rescue carbs;
Pump settings such as basal programs, I:C ratios, ISF, max/min settings;
Food diary.

Much of the present invention's historical data has come from Diasend, the Swedish data company. We have written a script that takes the Diasend Excel export format and automatically loads from a home computer into the database format in Minerva's Cloud. This script could be used to import data from any individual currently using Diasend. At present, we have the capability to import all data in real time from an internet connected pump and we have a large amount of data using that method. We also plan to license large historical data collections to verify and supplement our current algorithms.

The present invention aims to become an “Artificial Parent,” a hybrid of a decision support system and a closed loop system. This is a somewhat different approach from that of both the DIY community and the mainstream AP groups and may fill a niche as the T1D world awaits the perfect AP system.

Minerva already has in development a decision support system App that would serve well as a vehicle to disseminate our Big Data improved analyses to parents and patients. Even without real time input from a pump, we could give caregivers very helpful information and advise them. For example, we could post to the phone App the ISF for that time of day based on the last month of corrections in the historical data. Similarly, we could post the I:C based on all the post-meal corrections and rescue carbs for the prior month.

For those who have CGM and pump data in real-time we see a very sophisticated hybrid: Not only can Minerva post your average ISF and effective meal dosing for an averaged period, but it also can warn you of an impending low. But Minerva can do more than advise; it can close the loop. Minerva could, for example, give a correction dose every 2 hours based on all the recent ISF values for that time of day, the starting CGM and time after the meal. It could decrease the dose appropriately the second time around for stacking. The well-rested parent who has relied on Minerva could go back to manual or continue with automatic in the morning.

The present invention will absorb a collection of existing data into our current system, which makes Minerva a good environment in which to study other data collections and employ them for practical use.

The present invention comprises a system and method for management of diabetes. A preferred embodiment of the present invention includes a diabetes management application designed for operation on a handheld organizer, such as a cell phone or personal digital assistant device. The present invention is used to log blood glucose levels, insulin intake, and nutrition information (carbohydrates, protein, fiber, fat and calories), activity and notes. Based on user settings such as carbohydrates to insulin ratios for various kinds of meals, glycemic index composition, blood glucose target area, high/low glucose levels compensation through insulin and carbohydrates, and weight, the present invention computes the recommended dosage taking into consideration variables such as blood glucose level, activity, meals, etc. A food diary may be used to track post-meal glucose excursions. Given the fact that most people eat the same meal items regularly, it may be possible to use machine learning to correlate post meal glucose with food items and give dosing advice pre-meal once the menu is known. It may also be possible to use pictures of food to aid in automatic or recommended manual dosing. In some embodiments, it may also be possible to encourage people to make better food choices when a post-prandial high CGM is noted, and the prior meal items entered were not healthy choices.

The present invention preferably uses one or more reference databases for computing meal nutrition factors and/or the amount of calories burned during activities. A food database is preferably provided for use with a handheld device. The food database preferably comprises at least a portion of the USDA food nutrition facts database. The food database preferably includes the most used items from the USDA food nutrition facts database. A corresponding desktop application is also provided which preferably contains corresponding portions of the USDA food nutrition facts database which can be uploaded as needed to the handheld device. The database would be updated to include the most common meal items used by the individual, and may be automatically or manually updated.

In one embodiment of the present invention, the an amount of carbohydrates needed to compensate for the burned calories or recommends alterations in pump basal rates to account for the activity.

The present invention is preferably capable of synchronizing databases between a handheld device and a personal computer running the desktop application. Record 950 includes activity data and is stored in activity log. Besides the date/time field columns, record 950 includes a field column for each of duration, calories and activity. The duration field column is the duration of an activity of the patient measured in minutes. The calories field column is the number of calories expended by the patient during exercise. The activity field column is the number of calories expended by the patient while performing the activity. It may also be possible for this information to be automatically entered via a fit bit or motion detector.

It may be possible for the patient to store notes and images of food for future review. Since records are individual records containing multiple fields, the storage space required is minimized. The meal intake data may be based on foods entered by the patient. The meal intake data may be based on the total nutritional content of a meal. The meal intake data may be inputted by the patient choosing one or more food items from the external food database. A portion size may be inputted which corresponds to the amount of food ingested by the patient. The glucose data may be received from a CGM which monitors the blood/tissue glucose of the patient. The insulin intake data may be received from an insulin pump which distributes insulin subcutaneously. Insulin intake recommendations may be based on the food intake data. The food intake data may include at least one of carbohydrate intake data, fat intake data and protein intake data. Multiple insulin to carbohydrate compensation ratios may be stored based on a time and/or meal type deemed appropriate for the patient. The activity data may include calories burned by the patient during an activity. The carbohydrate intake recommendations and calorie intake recommendations may be based on the activity data. Some embodiments may include tracking other nutrient data such as fats, proteins, carbs, salt, and calories.

The present invention may be implemented with any combination of hardware and software. If implemented as a computer-implemented apparatus, the present invention is implemented by using means for performing all of the steps and functions described above.

The present invention can also include an article of manufacture (e.g., one or more computer program products, having, for instance, computer user media. The media has embodied therein, for instance, computer readable code means for providing and facilitating the mechanisms for the present invention. The article of manufacture can be included as part of a computer system or sold separately. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention.

Although the patient management system 10 is presently described in relation to managing a single patient possessing one or more patient-specific meters 14, it should be understood that the system 10 can be used for managing many patients, each possessing one or more patient-specific meters 14. More generally, any other element of the system 10 can be provided in any number as desired. As used herein, “patient-specific meter” is a broad term, and is used in its ordinary sense to refer to a meter which can be uniquely identified with a single patient. Other meters described herein, such as the hospital meter 18, may be used by multiple patients.

As used herein, “caregiver” is a broad term and is used in its ordinary sense to refer, without limitation, to any person authorized to participate in the treatment of a patient. Examples of caregivers include physicians, physician assistants, nurses, parents, guardians, family members, etc. The server, in the depicted embodiment, generally comprises and/or houses at least one patient database, a flexible database, and a processing center. (The Data flowing from the patient to at least one of the patient-specific meters, can generally include any one or combination of the following: (a) login information such as a username and password; (b) current time/date/day; (c) exercise data such as the type and/or duration of exercise engaged in or to be engaged in, and/or the time and date that the exercise was or will be engaged in; (d) the patient's current height, weight, and/or age; (e) diet information such as the type and/or quantity of food and/or drink consumed or to be consumed, and/or the time and date that the food and/or drink was consumed or will be consumed; (f) insulin consumption information such as the type and/or quantity of insulin consumed or to be consumed, and/or the time and date that the insulin was consumed or will be consumed; (g) material samples for analysis; and/or generally any other factors affecting glucose concentration.

Data flowing from the meter(s) to the server can generally include any one or combination of the following: (a) analyte-concentration measurement values (including, if desired, corresponding dates/times and meter identification information); (b) update status pertaining to the algorithm(s) employed by the meter(s) to calculate and/or measure analyte concentration, or to any other soft-ware/firmware employed in the meter(s) 14; (c) meter calibration status; (d) physical location of the meter(s) (e.g., as may be determined by a GPS system (not shown) built into the meter(s) 14); (e) patient exercise data as discussed above; (f) the patient's current height, weight, and/or age; (g) patient diet information as discussed above; (h) patient insulin consumption information as discussed above; (i) rate of use, and/or quantities used, of disposable sample elements (e.g. test strips).

Data flowing from the server 12 to the primary caregiver terminal 16 can generally include any one or combination of the following: (a) analyte-concentration measurement values (including, if desired, corresponding dates/times and meter identification information); (b) update status pertaining to the algorithm(s) or software/firmware employed in the meter(s) 14 as discussed above; (c) meter calibration status; (d) physical location of the meter(s); (e) patient exercise data as discussed above; (f) the patient's current height, weight, and/or age; (g) patient diet information as discussed above; (h) patient insulin consumption information as discussed above; (i) patient insurance information; G) patient disease level classification; (k) current standing orders pertaining to the patient's care and made by a caregiver; (l) rate of use, and/or quantities used, of disposable sample elements. Patient may also input their symptoms. The data may also comprise second-order information generated by the database(s) such as trends in analyte-concentration measurement values; the patient's exercise, diet, and/or insulin consumption history; and/or effects of exercise, diet and/or insulin consumption on patient analyte concentration. In various embodiments, the data may generally include any information be in one-way or two-way electronic communication, as further described herein, with the other elements of the system 10, such as the patient-specific analyte detection meter(s), the caregiver terminal, any hospital-based meter, any insurance-provider terminal, and/or any secondary-caregiver terminal.

Data flowing from the primary caregiver terminal to the server can generally include any one or combination of to measure a secondary analyte such as a ketone, HbAlC, etc.; (d) requests to take an additional measurement of a given analyte; (e) requests to visit the caregiver; (f) changes to patient disease level classification; (g) requests to start, adjust or terminate any one or combination of the patient's: diet, insulin consumption, exercise regimen, and/or analyte-concentration measurement schedule; (h) text messages (e.g., general medical advice, requests for additional information from the patient, or other treatment-related information) from the caregiver to the patient. In one embodiment, the caregiver can send any or all such information, requests, updates, etc. directly to the patient's phone, which can relay such information to the server for storage in the patient's database for any record-keeping or trend-tracking purposes as desired. Some embodiments may also include direct notifications to the application itself or direct contact to the patient such as email, text, or voicemail.

Data flowing from the server to the meter(s), including cell phone(s), can generally include any one or combination of the following: (a) updates or other modifications to the algorithm(s) or other software/firmware employed in the meter(s) as discussed above;
(b) commands to lock a patient out of an un-calibrated meter; (c) requests to measure a secondary analyte such as a ketone, HBAlC, etc.; (d) requests to take an additional measurement of a given analyte; (e) requests to visit the caregiver; (f) changes to patient disease level classification; (g) requests to start, adjust or terminate any one or combination of the patient's: diet, insulin consumption, exercise regimen, and/or analyte-concentration measurement schedule; (h) reminders to calibrate the meter(s); (i) calibration information; G) text messages (e.g., general medical advice, requests for additional information from the patient, or other treatment-related information) from the caregiver to the patient.

Data flowing from the patient-specific meter(s) to the patient can generally include any one or combination of the following: (a) analyte-concentration measurement values; (b) requests to measure a secondary analyte such as a ketone, HBAlC, etc.; (c) requests to take an additional measurement of a given analyte; (d) requests to visit the caregiver; (e) requests to start, adjust or terminate any one or combination of the patient's: diet, insulin consumption, exercise regimen, and/or analyte-concentration measurement schedule;
(f) reminders to calibrate the meter(s) 14; (g) calibration information.

Data flowing from the server to any insurance-provider terminal(s) may include any one or combination of the following: rate of use, and/or quantities used, of disposable sample elements; analyte-concentration trend information as may indicate patient compliance with a management plan; and/or information relating to the patient's general health.

As discussed above, an individual patient database may be provided, in certain embodiments, for each unique patient under management by the system. Thus, a plurality of patient databases may be stored in, and/or accessible by, the server. The individual patient databases can include a wide variety of information relating to a patient's medical condition, including any one or combination of the following: (a) analyte-concentration measurement values (including, if desired, corresponding dates/times and meter identification information);
(b) update status pertaining to the algorithm(s) or software/firmware employed in the meter(s) as discussed above; (c) meter calibration status; (d) physical location of the meter(s); (e) patient exercise data as discussed above; (f) the patient's current height, weight, and/or age; (g) patient diet information as discussed above; (h) patient insulin consumption information as discussed above; (i) patient insurance information; G) patient disease level classification; (k) current standing orders pertaining to the patient's care and made by a caregiver; (l) rate of use, and/or quantities used, of disposable sample elements. The database may also include second-order information generated by the data-base(s) such as trends in analyte-concentration measurement values; the patient's exercise, diet, and/or insulin-consumption history; and/or effects of exercise, diet and/or insulin consumption on patient analyte concentration. A caregiver can then sort and/or plot the stored measurement data according to one or more of these data types. For example, if a caregiver wishes to see the frequency of measurement as compared to a variation in analyte concentration, such a plot can be easily derived from the tabulated, stored data.

In one embodiment, the meter(s) communicate with the server via a connection to a personal computer, which is itself connected to the internet through any suitable data link. Alternatively, the analyte detection meters can be adapted to communicate directly with the server via a wireless connection such as a GSM or cellular network, or via the internet or other proprietary connections such as WiFi®, Bluetooth®, or other wireless data link. Generally, the data paths may comprise any suitable wired, cellular, or wireless data links, or combinations of wired and wireless data links.

The patient-specific analyte detection meter(s) are generally configured to measure a concentration of one or more analytes, such as glucose, associated with a diabetic condition. The meter(s) can comprise any suitable invasive, minimally invasive, or non-invasive analyte detection device or mechanism. Many such analyte detection systems are known, including electrochemistry-based detection systems, reflectance-spectroscopy-based detection systems, transmission-spectroscopy-based detection systems, etc. Other suitable detection systems include those configured to perform concentration measurements of multiple analytes.

The patient-specific meter(s) used in connection with the patient management system 10 described herein, may also be configured to store and process the resultant analyte concentration measurements. Thus, the meter(s) may include data storage and processing capabilities. Such data storage and processing capabilities can be provided by any suitable processor and storage medium. The meter(s) may be configured to store one or more software algorithms to perform functions such as manipulation or processing of the measurement data obtained by the meter as will be further described below. Thus, a portion of the data storage of the meter can be configured to include a “firmware” storage device which can be provided in addition to a measurement data storage device. Alternatively, a firmware package and the measurement data can be stored on a single piece of hardware. As used herein, the term “firmware” is a broad term and is used in its ordinary sense to refer, without limitation, to one or more strings of computer code which is stored in a read/write memory chip or other updatable data storage device capable of retaining one or more strings of computer code when a power source is disconnected from the device.

### Using Patient Data to Recommend Therapeutic Administration

Some embodiments may use patient history to estimate basal and bolus insulin dosages, which it recommends to the patient for manual administration. Some prior art connects to the patient's primary caregiver, who can record changes in treatment and customize the algorithm manually. Other prior art is more primitive and merely calculates recommended insulin dosage based on carbohydrates ingested. Some embodiments may include the creation or use of a more sophisticated interactive system where caregiver can manipulate data and make requests that a computerized system using algorithms, machine learning or AI which can then deliver pertinent clinical analyses and directions for improvement of patient's condition.

The present invention connects and/or CGM sensor. It takes the patient's CGM data and compares it to historical and other data in the program to decide if the patient's glucose levels are too high 207 or too low 208.

If the patient's insulin levels are too high 207, the present invention's program will calculate a recommended insulin dosage and recommend this to the patient. This may appear as a notification on an app, browser, and/or smartwatch and may notify the caregiver as well as the patient. The patient and/or caregiver will have the option to manually administer the recommended amount, which may be automatically or manually recorded in the program. The program may also accept other patient information, such as mood, body temperature, or other factors upon administration of insulin. In some embodiments, the patient or caregiver will also have the option to approve or cancel an automatic command from the server within a certain time period (Hybrid system) or can alternatively put the system on a complete automatic mode. The system may have an analogous program on the local pump or phone or handheld device which is updated regularly by the server using Cloud capabilities. The local program can be used should the internet or connectivity fail for some reason.

If the patient glucose levels are too low 208, the present invention will take patient history and machine learning 206 into consideration to recommend a decrement in pump insulin infusion or a carbohydrate dosage that will balance the patient's blood glucose without causing it to go too high. This dosage may be in grams, or may be given as a fraction of a pre-selected type of glucose tablet, which may be easier for children. This would likely be a flexible choice, where the patient and/or caregiver would select the type of carbohydrate or glucose supplement they prefer to use and the program calculates accordingly. The patient and/or caregiver will have the option to manually administer the recommended amount, which may be automatically or manually recorded in the program. The program may also accept other patient information, such as mood, body temperature, or other factors upon administration of carbohydrates. The program has a predictive aspect so that it can turn the pump off well ahead of time before a low is anticipated to occur

The patient or caregiver also has the option to input their own instructions into the program with set messages, i.e. If glucose is greater than 250, “please give an insulin correction of 60 mg/dl” or if glucose is below 60 “please give a juice box.” This may be useful early in the present invention's use with a patient before it has the chance to gather enough data for machine learning, or if the caregiver wants to make treatment simpler for a secondary caregiver such as a babysitter or school nurse.

The Diamon backend has continuously running processes that fetch the CGM data and compare the value to the current set of instructions to see if an abnormal glucose range is triggered. If “YES”, then an alert goes out with a question such as “Do you have symptoms?” Depending on the answer, Diamon sends the parental instruction for that situation and the youth presses “DONE” when accomplished. Everyone sees the “DONE”. Then, there is no need to call school if you are the parent. No need to check the schoolyard if you are the school nurse.

As Diamon moves from step to step within the tree it functions like a “state machine” with a final state of “All Responses Received.” A change in glucose range can interrupt the state; no response within a certain time triggers a repeat alert. The current alert cycle is visible on the “Diamon Alert Page” of the app. The alert might show, for example, that a “Low” occurred 10″ ago, the youth had no symptoms but took 4 glucose tabs, and that Diamon will send out a recheck alert in 5″. All old cycles will be logged below in the “Alert History.”

The program may also be designed to incentivize good behavior in patients, particularly children, but gamifying their diabetes treatment. Fast and accurate responses to the program's prompts may earn them points or other rewards, which may or may not have real-life or social rewards involved. These rewards may be optimized and personalized via machine learning. There may also be leaderboards with real time data from participating users, and competitions where users can compete to improve their treatment e.g. time to dose after high or time to take carbs after lows, average CGM, least CGM variability, best night control, etc.

Users may also be provided with educational materials and given pop quizzes, a “daily diabetes quiz,” or be otherwise tested. The invention envisions the capability for the CGM to report its readings to the Internet using a secured communication protocol (for example with HIPPA compliance) such that the data can be analyzed by a cloud-based machine learning algorithm or a fixed algorithm which can offer predictive analytics and optimization of therapeutic timing and dose delivery of insulin as well as optionally other therapeutics and/or recommendations (e.g., delivery of glucagon, or other naturally occurring or synthetic mediators as well as diet recommendations for consumption of specific diet composition (e.g., carbohydrate composition, sugar, fat as well as quantity as well as recommendations regarding consumption of fluids). The machine learning can be a blend of aggregate data with personal historical data such that this blended data is optimized to provide the best recommendations to improve glucose control.

Some embodiments may watch, allow patient to control pump via smart watch (convenient in restaurants), and/or have a smart watch and/or pump alarm while sleeping to alert patient and/or caregiver if insulin or carbohydrates need administered.

Some embodiments may allow patients and caregivers to record dietary information as part of a post prandial dietary journal. This system takes dietary journal info which confirms diet information by ‘asking’ when CGM data sends an ‘alert’ that blood sugar has increased, so that the system triggers the end-user to reply with what and when food was eaten. The system will analyze the composition and quantity of the food and will make prescriptive recommendations for what should and should not be consumed to optimize blood sugar.

The diabetes management system computes and recommends amounts of carbohydrates and insulin that the diabetes patient should consume based on types of data inputted by the diabetes patient via a user interface (UI) presented by the phone or PDA. In one preferred embodiment of the present invention, a desktop application is provided as a counterpart to an application running on a handheld device. The desktop application preferably contains all of the functionalities of the handheld application along with extended reporting and database maintenance capabilities. The desktop application may use an Access database structure and ActiveX data objects database connections or an online portal of another kind. Preferably, this will sync with the insulin pump and app in real-time or at otherwise reasonable intervals.

The present invention is preferably also designed to provide for ready integration with insulin pumps using, for example, an infrared link. Diamon has already integrated with one pump on the market via Bluetooth communication, and this pump is capable of providing insulin data in real time to Minerva's (the inventor's) servers. Preferably, the present invention can download and centralize information regarding insulin boluses, daily totals history, alarm history—with a description for each code, actual settings and programs, and the like. The present invention preferably also can download information from any number of glucometer devices. The present invention also preferably provides easy data entry for users, allowing a user to focus on recording of meals, activities and important events.

Preferably, data can be downloaded from the hand-held device to one or more databases operating in connection with the desktop application. A user preferably is able to analyze data downloaded to the desktop application using a number of tabular listings, linear graphs, bar graphs and pie charts. Preferably the user is also able to “pack” or packetize data, for example, for a certain period or periods of time. The data can then be conveniently transmitted to a physician for analysis. The present invention is also prefer-ably capable of synchronizing multiple handheld devices on a single desktop computer.

In operation, a user is preferably capable of selecting one or more items through the desktop application for uploading to a handheld device from, for example, the food database and/or the activity database. Additionally, a user can select from a full list of items provided from the USDA database as well as from updates provided by a third party or new items entered by the user. According to one embodiment of the invention, a method of remotely managing a diabetic condition comprises configuring a meter to calculate a correction bolus using a correction algorithm. The method further comprises configuring the meter to measure a concentration of an analyte in a material sample from a patient. In some embodiments, the analyte to be measured is an indicator of a diabetic condition. A result of the measurement is a variable used in the correction algorithm. Some embodiments of the method further includes providing a central server in two-way communication with the meter and configuring the server to store information received from said meter. The method can also include allowing a medical caregiver to access the central server using a terminal in two-way communication with the central server; and further allowing the medical caregiver to modify the correction algorithm in the meter via the terminal.

According to another embodiment of the invention, a system for allowing a medical caregiver to remotely manage a diabetic condition of a patient is provided. The system comprises a central server containing a database of information associated with at least a first patient and a meter associated with the first patient. The meter is adapted to calculate a correction bolus using a correction algorithm and a plurality of patient-affected and caregiver-affected variables. The meter is also in two-way communication with the central server. A terminal is associated with the caregiver and is adapted to allow the caregiver to view the database associated with at least the first patient. The terminal is further adapted to allow the caregiver to modify the correction algorithm in the meter associated with the first patient.

In yet another embodiment of the invention, a system for managing a diabetic condition of a patient comprises a handheld meter having a digital memory and computing abilities. The digital memory is programmed with a correction algorithm for calculating a correction bolus from a plurality of parameters. A central server is in two-way communication with the meter. At least some of the parameters for calculating the correction bolus are stored in and updatable by said central server. The central server is configured to allow a care giver to remotely modify at least one of said parameters for calculating a correction bolus. In some embodiments, the functions of the server can be implemented in the meter itself, thereby eliminating the need for a central server as a separate element in the network. In embodiments of a de-centralized patient management system, a plurality of system elements can communicate with one another according to various peer-to-peer or other de-centralized protocols.

In certain embodiments, the patient management system generally includes a number of elements, such as a central server, at least one patient-specific analyte detection meter, and at least one terminal 16 associated with a caregiver. Some embodiments may further comprise a hospital-based analyte detection meter, an insurance-provider terminal, and/or a secondary-caregiver terminal. Of course, the system may include additional elements as desired. Alternatively, certain elements or various sub-combinations of these elements can be used independently of the other elements of a patient management system.

Although the patient management system 10 is presently described in relation to managing a single patient possessing one or more patient-specific meters, it should be understood that the system can be used for managing many patients, each possessing one or more patient-specific meters. More generally, any other element of the system can be provided in any number as desired. As used herein, “patient-specific meter” is a broad term, and is used in its ordinary sense to refer to a meter which can be uniquely identified with a single patient. Other meters described herein, such as the hospital meter 18, may be used by multiple patients. As used herein, “caregiver” is a broad term and is used in its ordinary sense to refer, without limitation, to any person authorized to participate in the treatment of a patient. Examples of caregivers include physicians, physician assistants, nurses, parents, guardians, family members, etc.

The server generally comprises and/or houses at least one patient database, a flexible database, and a processing center. The server is preferably configured to be in one-way or two-way electronic communication, as further described herein, with the other elements of the system, such as the patient-specific analyte detection meter(s), the caregiver terminal, any hospital-based meter, any insurance-provider terminal, and/or any secondary-caregiver terminal.

Data flowing from the patient to at least one of the patient-specific meters, can generally include any one or combination of the following: (a) login information such as a username and password; (b) current time/date/day; (c) exercise data such as the type and/or duration of exercise engaged in or to be engaged in, and/or the time and date that the exercise was or will be engaged in; (d) the patient's current height, weight, and/or age; (e) diet information such as the type and/or quantity of food and/or drink consumed or to be consumed, and/or the time and date that the food and/or drink was consumed or will be consumed; (f) insulin consumption information such as the type and/or quantity of insulin consumed or to be consumed, and/or the time and date that the insulin was consumed or will be consumed; (g) material samples for analysis; and/or generally any other factors affecting glucose concentration.

Data flowing from the meter(s) to the server can generally include any one or combination of the following: (a) analyte-concentration measurement values (including, if desired, corresponding dates/times and meter identification information); (b) update status pertaining to the algorithm(s) employed by the meter(s) to calculate and/or measure analyte concentration, or to any other soft-ware/firmware employed in the meter(s); (c) meter calibration status; (d) physical location of the meter(s) (e.g., as may be determined by a GPS system (not shown) built into the meter(s)); (e) patient exercise data as discussed above; (f) the patient's current height, weight, and/or age; (g) patient diet information as discussed above;
(h) patient insulin consumption information as discussed above; (i) rate of use, and/or quantities used, of disposable sample elements (e.g. test strips).

Data flowing from the server to the primary caregiver terminal can generally include any one or combination of the following: (a) analyte-concentration measurement values (including, if desired, corresponding dates/times and meter identification information);
(b) update status pertaining to the algorithm(s) or software/firmware employed in the meter(s) 14 as discussed above; (c) meter calibration status; (d) physical location of the meter(s); (e) patient exercise data as discussed above; (f) the patient's current height, weight, and/or age; (g) patient diet information as discussed above; (h) patient insulin consumption information as discussed above; (i) patient insurance information; G) patient disease level classification; (k) current standing orders pertaining to the patient's care and made by a caregiver; (l) rate of use, and/or quantities used, of disposable sample elements. The data may also comprise second-order information generated by the database(s) such as trends in analyte-concentration measurement values; the patient's exercise, diet, and/or insulin-consumption history; and/or effects of exercise, diet and/or insulin consumption on patient analyte concentration. In various embodiments, the data may generally include any information contained in the patient database. The system may permit the primary caregiver to request specific data (such as any of the data) at specific times, or alternatively a previously specified set or type of data selected from the data can be “pushed” to the primary caregiver at regular time intervals as desired.

Data flowing from the primary caregiver terminal to the server can generally include any one or combination of the following: (a) updates or other modifications to the algorithm(s) employed by the meter(s) to calculate and/or measure analyte concentration, or to any other software/firmware employed in the meter(s); (b) commands to lock a patient out of an un-calibrated meter; (c) requests to measure a secondary analyte such as a ketone, HBAlC, etc.; (d) requests to take an additional measurement of a given analyte; (e) requests to visit the caregiver; (f) changes to patient disease level classification; (g) requests to start, adjust or terminate any one or combination of the patient's: diet, insulin consumption, exercise regimen, and/or analyte-concentration measurement schedule; (h) text messages (e.g., general medical advice, requests for additional information from the patient, or other treatment-related information) from the caregiver to the patient. In one embodiment, the caregiver can send any or all such information, requests, updates, etc. directly to the patient's meter(s) and the meter(s) can relay such information to the server for storage in the patient's database for any record-keeping or trend-tracking purposes as desired.

Data flowing from the server to the meter(s) can generally include any one or combination of the following: (a) updates or other modifications to the algorithm(s) or other software/firmware employed in the meter(s) as discussed above; (b) commands to lock a patient out of an un-calibrated meter; (c) requests to measure a secondary analyte such as a ketone, HBAlC, etc.; (d) requests to take an additional measurement of a given analyte; (e) requests to visit the caregiver; (f) changes to patient disease level classification; (g) requests to start, adjust or terminate any one or combination of the patient's: diet, insulin consumption, exercise regimen, and/or analyte-concentration measurement schedule; (h) reminders to calibrate the meter(s); (i) calibration information; G) text messages or notifications (e.g., general medical advice, requests for additional information from the patient, or other treatment-related information) from the caregiver to the patient.

Data 32 flowing from the patient-specific meter(s) to the patient 22 can generally include any one or combination of the following: (a) analyte-concentration measurement values; (b) requests to measure a secondary analyte such as a ketone, HBAlC, etc.; (c) requests to take an additional measurement of a given analyte; (d) requests to visit the caregiver; (e) requests to start, adjust or terminate any one or combination of the patient's: diet, insulin consumption, exercise regimen, and/or analyte-concentration measurement schedule; (f) reminders to calibrate the meter(s); (g) calibration information.

Data flowing from the server to any insurance-provider terminal(s) may include any one or com-bination of the following: rate of use, and/or quantities used, of disposable sample elements; analyte-concentration trend information as may indicate patient compliance with a management plan; and/or information relating to the patient's general health.

Intensive insulin therapy has been the ideal standard for diabetes care over the past 25 years. Currently, patients using the PITA approach to manage their disease must rely on written forms for dosage calculation and record keeping. Electronically integrating the patient's preferences and data into this device greatly enhances the precision, accuracy, reliability and ease of use of the approach. The device also assists in the preparation of patients for greater self-management of their diabetes.

Vesting greater control over the treatment of diabetes in the patient does not eliminate the need for periodic review and, if necessary, intervention by physicians and diabetes educators. The capacity of device to transmit data to the clinic of the patient's health provider, however, contributes to efficient and therapeutically sound management of diabetes. Competent health professionals at the receiving end of the data can monitor the extent to which the patient is competently employing the approach and, if necessary, suggest changes to the technology, which will ultimately improve patient-provider communication and interaction without adding a significant added cost or time burden.

The program could be integrated with diabetes health care providers and/or their own programs and systems. It may include a personalized back up help menus for separate physician and physician group practices, an online diary of basal programs, I:C values, etc for individual users which could be shared with providers, and/or thus become a “one-stop-shop” for diabetes practices as a patient interface.

Some of the operations may be omitted, further operations may be included, and the operations may be performed in temporal orders different from those shown. Operations may be performed by a health-care professional and/or by the manufacturer or distributor of a dedicated device or application software. The system maker designs the basic personal insulin treatment algorithm (PITA), specifying which factors are included and coding the necessary calculations and data input/output routines. The system maker may also generate one or more templates 213 from the design, to provide an interactive program for a health-care professional to enter data associated with particular patients.

The patient's medical history data may arise from interviews with the health-care professional, from laboratory tests, and/or from data uploaded from a device such as that described above. Workup and algorithm selection blocks may occur at the professional's office or similar location. Data loading may occur in the office or over a communications facility such as the Internet.

The remaining operations may be carried out by a patient's device such as 100, at any convenient location, including where the medication is to be administered. Dose administration is normally performed by the patient from an evaluated-dose display, although the device could be integrated with an insulin pump or other modality for direct infusion. Data-request blocks allow the patient or the device itself to choose. to perform certain operations, such as entering variations in food intake for a meal. Carbohydrate amounts entered at 223 can then be used to alter subsequent dosage calculation. Other patient history could be entered in the same manner, if desired. For example, separate urine ketone test results could be entered here. It may transmit this reading and or others. Other operations may include sounding alarms, signaling clock time, and other events.

Operations determine a contemporaneous dosage when requested. It employs the personalized or management plan to calculate a dose of a medication such as an insulin bolus from history data. The history data stores patient history data, such as current and past blood-glucose readings, recorded carbohydrate intakes, and previous insulin dosages, for calculating dosage data. Patient history data could also be down-loaded into the health-care professional's medical data for usages such as selecting or modifying the parameters. It evaluates the calculated dosage according to limit values, etc., for safety purposes. It displays or otherwise advises the patient of the dosage amount and requests confirmation. For most present administration modalities, the patient manually administers the medication. However, it may administer the medication automatically upon confirmation through an insulin pump or other means.

An electronic template according to the invention could employ an interactive template having a layout similar to this form for entry of individualized patient data. Such a template could be custom-coded for this application, or might be implemented with a conventional spreadsheet application program. The foregoing description and drawings illustrate specific embodiments of the invention sufficiently to enable those skilled in the art to practice it. Other embodiments may incorporate structural, logical, electrical, process, and other changes. Examples merely typify possible variations. Individual components and functions are optional unless explicitly required, and the sequence of operations may vary. Portions and features of some embodiments may be included in or substituted for those of others. The scope of the invention encompasses the full ambit of the following claims and all available equivalents.

There are many possible sources of information input into the program. The diabetes-related information collected may include calendar information from a calendar application on mobile device, which would log “cheat days” as well as weekly meetings/dinner plans and take these into account. According to some embodiments calendar information may include an appointment scheduled near when a blood glucose measurement was received (e.g., a soccer practice within 30 minutes). Diabetes-related information may include activity information from an activity tracking application on mobile device. In some embodiments activity information may include an activity level near when the blood glucose measurement was received (e.g., a pedometer application indicating that the patient just walked an abnormally long distance). Diabetes-related information may include nutrition information from a nutrition tracking application on mobile device. Nutrition information may include food or drink consumed near when a blood glucose measurement was received. Diabetes-related information may include image recognition information from an image recognition application on mobile device (e.g., recognition of a particular fast food restaurant's logo in a recently taken photo). Some embodiments may include the recognition of a particular fast food restaurant from the GPS of user. This might prompt a food database to open that was specific for that fast food restaurant. Diabetes-related information may include mobile device connectivity information from mobile device.

Mobile device connectivity information may include an indication that the mobile device has connected with another electronic device (e.g., a bicycle sensor, a vehicle electronics system, a router, etc.) via Wi-Fi or via short-range wireless communication protocol. Diabetes-related information may include location information, such as GPS coordinates. Diabetes-related information may include an elapsed time since diabetes management application received a previous blood glucose measurement from a patient.

According to some embodiments, diabetes-related information includes patient-provided answers to a context-related question subset. In some embodiments, the context-related information includes patient-provided answers to one or more context-related questions. In some of these embodiments, the patient-provided answers include an explanation of the previous frequency of blood glucose measurements of the patient. Further information relating to context-related questions are described further herein.

Diabetes management application may include one or more predetermined prompt criteria. Predetermined prompt criteria may differ based on the type of diabetes-related information. For example, diabetes-related information received may include calendar information from the calendar application on a mobile device. In these embodiments, the one or more predetermined prompt criteria may include a calendar appointment type scheduled for a specified amount of time of a specific current time. In these embodiments, the one or more predetermined prompt criteria may include a calendar appointment type comprising an appointment involving an elevated level of physical activity compared to a normal physical activity level for the patient.

The diabetes-related information received may include activity information from the activity tracking application on a mobile device. In these embodiments, the one or more predetermined prompt criteria can include normal and elevated physical activity levels for the patient. The diabetes-related information may include nutrition information from the nutrition tracking application on a mobile device. In some of these embodiments, the one or more predetermined prompt criteria includes normal and elevated nutritional glucose levels of the patient. The diabetes-related information may include image recognition information from the image recognition application on a mobile device. According to some of these embodiments, the one or more predetermined prompt criteria includes an image-based diabetes risk factor. The diabetes-related information may include mobile device connectivity information. In these embodiments, the one or more predetermined prompt criteria can include an indication that the mobile device has connected with another electronic device.

Received diabetes-related information may include the elapsed time since diabetes management application received a previous blood glucose measurement from the patient. In some of these embodiments, the one or more predetermined prompt criteria comprises a specified frequency of blood glucose measurements. The specified frequency of blood glucose measurements may be tailored to the patient based on previous blood glucose measurements of the patient, previously collected context-related information, a previous frequency of blood glucose measurements of the patient, or combinations thereof. Whether or not diabetes-related information includes a specified frequency, diabetes management application may identify the appropriate predetermined prompt criteria.

Diabetes management application may make a comparison of the received diabetes-related information to one or more predetermined prompt criteria. In some embodiments, diabetes management application may identify a blood glucose management action prompt condition. A prompt condition may be identified based on the comparison of diabetes-related information to predetermined prompt criteria. For example, if diabetes-related information includes calendar information and predetermined criteria includes a calendar appointment type, diabetes management application may analyze the received calendar information to determine whether it includes calendar appointments of the specified type.

Diabetes management application may generate a blood glucose management action prompt. A prompt may be based on the blood glucose management action prompt condition. In some embodiments, diabetes management application presents the blood glucose management action prompt to a patient via the user interface of a mobile device. The blood glucose management action prompt may comprise a prompt to take a blood glucose measurement, a prompt to take in carbohydrates, a prompt to take in insulin, a prompt to exercise, or any combination thereof. The app may also learn regular exercise patterns or sense exercise and adjust accordingly. Prompts may be presented using a prompt tone based on patient age or maturity level. Using different tones based on the patient may be utilized to increase patient engagement.

Mobile device may receive a blood glucose measurement from a patient. In some embodiments, a patient performs a blood glucose measurement using blood glucose monitor. The blood glucose measurement may be received from blood glucose monitor at diabetes management application on mobile device. The operation of the blood glucose monitor and its connections to the mobile device are detailed further herein.

In some embodiments, the system can collect context-related information concerning the blood glucose measurement. The context-related information can provide context for the corresponding blood glucose measurement, which can allow for more informed diabetes management decisions and actions, along with better overall diabetes care. Context-related information may be collected with the diabetes management application 30. Diabetes management application may collect context-related information from a variety of sources, including by using components and functions of mobile device.

The context-related information collected may include calendar information from a calendar application on mobile device. According to some embodiments calendar information may include an appointment scheduled near when a blood glucose measurement was received (e.g., a lunch scheduled at a particular restaurant). Context-related information may include activity information from an activity tracking application on mobile device. In some embodiments activity information may include an activity level near when the blood glucose measurement was received. Context-related information can also include nutrition information from a nutrition tracking application on mobile device. Nutrition information may include food or drink consumed near when a blood glucose measurement was received. Context-related information may also include image recognition information from an image recognition application on mobile device. Context-related information can include mobile device connectivity information from mobile device.

Mobile device connectivity information may include an indication that the mobile device has connected with another electronic device via Wi-Fi or via short-range wireless communication protocol. Context-related information may include location information, such as GPS coordinates.

According to some embodiments, context-related information includes patient-provided answers to a context-related question subset, or combinations thereof. In some embodiments, a context-related question subset is selected based on the received blood glucose measurement of the patient. In some embodiments, a context-related question subset is selected using a frequency of receiving blood glucose measurements of the patient. A context-related question subset may be selected based on a time of day of the received blood glucose measurement, or in some embodiments, based on previous answers to context-related questions and/or previous blood glucose measurements.

Context-related questions may include health- and behavior-related questions. Context-related questions may include questions related to the diet, behavior, health, and/or symptoms of the patient. Context-related questions may include questions about the frequency of receiving blood glucose measurements of the patient and/or time of day of the received blood glucose measurement. Examples include:
Have you checked your feet today?
Do you have any sores on your feet?
Have you had a professional check your feet? Have you had a professional check your eyes? Have you had diabetes education?
Have you lost consciousness or don't recall a period of time due to blood sugar?
Are you struggling to keep your blood glucose in control?
Are you struggling to count carbs?
Are you struggling to calculate insulin amounts?

In some embodiments, the context-related question subset includes a personal question unrelated to health measurements. Questions may be presented using a question tone based on patient age or maturity level. Including questions unrelated to health measurements and using different tones based on the patient may be utilized to increase patient engagement. The patient may also be offered educational games on their mobile device, and questions may be phrased in a child-friendly manner if the patient is of a certain age.

Personal questions may relate to activities or items that the patient enjoys. For example, a question may be presented on whether a user has a pet or has a favorite sports team. Future questions or content may be added based on answers to personal questions. For example, future questions may inquire into a pet's name if a patient indicates that he or she has a pet. Similarly, future questions may ask about recent games of a favorite sports team.

The tone of questions may be varied based on the patient's age or maturity level. For example, for young children simple sentence structures may be used and the tone of the questions can become friendlier or more enthusiastic. Conversely, for an older patient or patient who wishes to have a more “removed” experience, formal language and forms of address may be used.

Diabetes management application may perform the process of packaging the collected context-related information with the blood glucose measurement of the patient. The packaged context-related information and blood glucose measurements of the patient may be provided to a server. The diabetes management application may provide the packaged context-related information to server. Server may be remotely located.

In some embodiments, mobile device includes or utilizes a user interface. User interface may request that the patient connect via Bluetooth to a device. The patient can press the “Connect Bluetooth Device” button to activate the connection. When the patient activates this connection, a signal representative of the blood glucose measurement can be transmitted to the mobile device. Diabetes management application can then run an algorithm to analyze the signal

Server may receive information from diabetes management application. In some embodiments, server may receive packages of patient diabetes information from a plurality of patients. In these embodiments, each package of patient diabetes information may be provided by a diabetes management application on a mobile device of a patient.

In many embodiments, each package of patient diabetes information comprises a blood glucose measurement and context-related information of a patient. Server may store packages of patient diabetes information from a number of patients, for example from a patient number 1 to a patient number NN. The server may store multiple packages of patient diabetes information for each patient, for example from package number 1 to package number nn. Patients may have differing amounts and types of information. It will be understood by practitioners learned in the art that packages of data may be stored in a number of manners, including by utilizing a database or other storage system. Patient diabetes information may be processed or stored in ways other than or in addition to using packages or in order of package receipt.

In many embodiments, the diabetes management application can send additional information with the context-related information and the blood glucose measurement, such as the time of day, activity level, GPS location, weather, home automation data, shopping lists and other information created by connected personal assistants such as Amazon Echo, and other information. In some situations, such additional information can constitute context-related information. This transmission can be via Wi-Fi, telephone connection, or other suitable communication channel. The transmission can be in an encrypted form. The ACC server can verify the transmitted information according to the patient's serial number, index the information to the patient, and store the information according to the patient's clinic on server databases. In some systems, the diabetes management application can gather the blood glucose measurement, the context-related information, and the above-referenced additional information from the patient and can function independently of any ACC server to help the patient manage his/her blood glucose levels.

The diabetes management application can display messages, text, images, and/or other content to the patient based on input provided by the patient. Examples of displays to patients may include educational information, advertisements, alerts, and rewards. Displays may be personalized based on received information. Examples of information from which displays may be personalized include the collected context-related information or previously collected context-related information. Displays may also be personalized based on current received blood glucose measurements and previous blood glucose measurements of the patient. Displays may also be based on any combination of this information.

One type of display is educational information for the patient on various topics. Examples of such educational information include when or how often to take blood glucose measurements, when or how often to eat or exercise, when or how much insulin or medications to take, and links to additional online health information.

A type of display may include tailored advertisements and coupons related both to specific healthcare items (e.g., drugs, lotions, fitness items, etc.) and to general items (e.g., food products). Advertisements may be based on information obtained from blood glucose monitor, such as information that blood glucose monitor has a low number of items used for testing blood glucose levels. One type of display is rewards, displaying in-application rewards granted to patient based on positive patient behaviors. The server can include functions to handle a rewards program. Upon patients submitting data to server, server can determine whether certain conditions have been met (e.g., submitting timely measurements, engaging in positive behavior, achieving positive measurements, giving positive answers to the context-specific questions posed by the diabetes management application, etc.). Upon a condition being met, server can add set levels of points to the patient's rewards account. The server can store the rewards points information for each patient and additionally send information to the diabetes management application indicating points and rewards achieved. Upon achieving a certain number of points, patients can be eligible for certain rewards, or may trade the points in for rewards. The rewards can include coins or prize money tied to the diabetes management application. The program may also include diabetes education games that give users rewards for understanding their diabetes.

### Using Patient Data to Automatically Administer Therapeutic

Some embodiments may use past patient data to administer treatment based on predictive algorithms, and can take into account external factors such as time of day when deciding an acceptable blood glucose range. Some embodiments may include systems and methods for diabetes management with automated basal and manual bolus insulin control including a delivery device, glucose sensor, and controller. A model predictive algorithm based on the user's physiological data is used to calculate a basal insulin dose, which is administered automatically. The user administers the bolus dose manually, though this is also calculated by a model predictive control algorithm.

The present invention connects a CGM sensor 201 as well as other sensors 101 to the network 205 where it is processed and then to the patient's insulin pump 201. Information can flow from the network 205 to the insulin pump 203 as well. The network intakes caregiver instructions 204 and patient history and machine learning 206 which may also include data from other patients of the invention as well as larger databases. Minerva's servers, which may host the present invention's machine learning capabilities, consider these factors and found patterns when patient data is received from their insulin pump and/or CGM sensor. It takes the patient's CGM data and compares it to historical and other data in the program to decide if the patient's glucose levels are too high 207 or too low 208.

If the patient's insulin levels are too high 207, the present invention's program will calculate a recommended insulin dosage and recommend this to the patient 209. This may appear as a notification on an app, browser, and/or smartwatch 211 and may notify the caregiver as well as the patient. There may be an option to have the patient's insulin pump automatically administer medication based on the program's calculations. This option would likely only become available once a certain threshold is met, such as a particular amount of data collected, time of use, or when the program can calculate an effective therapeutic within a pre-selected degree of confidence, or the patient or caregiver may be able to select automatic calculations from the first day of use. The program may also accept other patient information, such as mood, body temperature, or other factors upon administration of insulin or independently to aid in its calculations. It is also possible that the patient's bolus and basal dosages will be calculated separately with one administered automatically and the other manually.

If the patient glucose levels are too low 208, the present invention will take patient history and machine learning 206 into consideration to recommend a carbohydrate dosage that will balance the patient's blood glucose without causing it to go too high. This dosage will likely need to be manual, but there is the possibility for automatic administration of glucagon or automatic administration or sugars in a manner similar to total parenteral nutrition or otherwise if technology changes.

Some embodiments may allow the pump to make an automatic glucagon injection in case of emergency (would require alteration to the physical pump or possibly another device or patch). Glucagon may also be injected when the app is receiving very low or no data, there is no movement by watch, ring, or phone monitor, no answer from alarm clock. The app may send a message to the wearable before glucagon injection, and might call 911 or other listed numbers of friends and family if there is no response.

When analyzing data, the program may group users based on how they respond to insulin and carbohydrates and prioritize data from the patient's group in making predictions. Patients in a group may also have other commonalities—age, weight, etc. Other embodiments may, if the user is still for too long, send a notification to their watch or vibrate or sound an alarm from their watch of phone. If they do not respond three times (or any other set number of times), send a notification to caregiver and/or dial emergency services. Some embodiments may send text or phone push notifications when therapeutic is administered, and have an easy-access emergency stop button either physically or in the user's phone app, watch, or web browser, or another means of access. Some embodiments may send an emergency notification in case of abnormal reaction to insulin.

The app may be capable of using known history of meal times and usual carb contents to calculate required treatments, though this might require some manual input at the beginning. It may also be possible for the app to log information from uploaded pictures of food, or could even include a photo-gallery of meals with estimated carbs and then post-meal CGM traces for adjustment of algorithm. User might be able to set quick buttons on app for commonly eaten meals, with usual grams of carbs. If post-meal trace is off, app asks for item/carb content so as to improve future dosing. It may also be possible for the app, with a sync to another wearable or even to another phone feature, to note hand or jaw movement that suggests user is eating, then ask for user to log meal information and dose accordingly. The app may also ask if regular dinner and then dose accordingly, possibly leaving time or a prompt for manual cancellation.

User personalization may help the program dose appropriately. They may input, or the program may learn post-meal CGM traces for food items and combinations. User may input estimated insulin to carb ratios for those food traces with displays of optimal traces, menu items and dosing patterns. The program may also include insulin sensitivity readouts for seasons, time of day, days with heavy exercise, etc. and may even provide displays with comparisons of similar individuals in terms of age, weight and time since diabetes diagnosed. In addition to dosing the user with insulin, it may also give individualized advice on how to improve or better manage their blood sugar levels.

Data sources may include SMBG, CGM, insulin delivery data (MDI and CSII), and other BSN data inputs such as heart rate, body accelerometer data, blood pressure, respiration, EKG data, etc. Depending on data availability (intermittent or continuous, blood glucose alone, or a multivariate data stream), the present invention may provide different types of services that can be generally classified as:
Local Services: Applications that run on a portable device (e.g. a cell phone or tablet computer) communicating with an array of self-SMBG monitoring and CGM devices, an array of insulin delivery devices, and with other sensors in a BSN. The local service of DiAs is equipped with intelligent processing to provide an array of patient services, including safety supervision, local alerts, patient advisory functions, and closed-loop control (described further below);
Global Services: A centralized server communicating with multiple local services to provide different levels of data processing, advice, and training to patients; enable remote monitoring of glucose control profiles (e.g. parents monitoring remotely their children with diabetes); enable global alerts (e.g. a 911 call with GPS service to pinpoint a patient in need of emergency assistance), and to provide a physician-oriented information service presenting key data for multiple patients at a glance.

Incoming data are directed to a processing program and/or server which assesses the frequency, dimensionality, and quality of the incoming data. Based on the assessment, the DAC recommends different classes of data processing algorithms for the incoming data. Many of these algorithms already exist and are generally known, and can be classified as follows:

SMBG: This is currently the most established algorithmic class, including methods for the retrieval of SMBG data, evaluation of glycemic control, estimation of the risk for hypoglycemia, and information displays.

CGM: Key elements applicable to these methods have been defined. Most of the methods applicable to CGM alone have extensions capable of dealing with input/output to/from an insulin pump.

Other: Heart rate changes can be used to indicate periods of physical activity, and more specifically periods of increased insulin sensitivity associated with exercise. These data inform diabetes control at several levels, including risk assessment for hypo-glycemia and closed-loop control.

The first step of data processing is estimation, given available data and using one of the methods described above. The state estimation results in assessment of the patient's risk status, which can be based on the risk analysis metrics presented in the background discussion above, and on biosystem observers or sensors, which process physiologic (and possibly behavioral) data to produce quantitative bio-system state estimators. These algorithms or methods are based on underlying mathematical models of the human metabolism and a Kalman filter, which produces system state estimation. Each system state estimator is a physiological or behavioral parameter of importance to the functioning of a person. The ensemble (vector) of biosystem estimators for a particular person represents the status of this person in terms of the blood glucose trend, availability of insulin, and risk for hypoglycemia. In essence, biosystem observers personalize the metabolic observation to a specific subject and extract 5 composite information from the vast array of raw data that allows the precise evaluation of the subject's condition. It is anticipated that the biosystem observers will reside within a wearable system, while their summarized output will be sent to both the local predictive and control algorithms or methods and to remote observers.

The primary output from the Patient State Estimation will be assessment of the patient's risk status for hypo- or hyperglycemia, based on the risk analysis and the LBGI/HBGI. If the data quality and density is adequate for the risk status of the patient (e.g. the patient is in a steady state performing regular SMBG resulting in LBGI and HBGI lower than certain preset thresholds), then program refers the data to algorithms that maintain the current patient status or fine-tune the patient's glycemic control. These algorithms can work in either an advisory or automated (closed-loop control) mode as follows:

If the data quality and density is inadequate for the risk status of the patient (e.g. the patient is at high risk for hypoglycemia, hyperglycemia, or both as indicated by the LBGI and HBGI exceeding certain preset thresholds), then:

In advisory mode, the program recommends enhanced monitoring (e.g. more frequent SMBG or switching to CGM for a certain period of time);

In automated control mode, the program switches the monitoring device to higher frequency SMBG measurement or to CGM mode (Note: such flexible monitoring devices are not currently manufactured, but are anticipated to be available in the future). Central to this architecture is the state estimator, which is the hub for exchange
of data between the program's monitoring devices and algorithmic services or related methods. The Biometric State Estimator may also exchange data with remote physicians and/or patient care centers over the Internet through a network interface;

The inputs used for state estimation are provided by various peripheral devices that monitor blood glucose fluctuations (SMBG Service, CGM Service), execute insulin delivery (Pump Service), or monitor other physiological parameters (Heart Rate Service, Esc. Service).

In turn, the state estimator provides feedback to these devices as determined by a Safety Service, which assesses the integrity of the received data and judges whether the peripheral input/output devices are functioning properly. Methods employed by the Safety Service include previously introduced detection of CGM sensor errors or judging the safety of insulin delivery;

Applications may include various advisory and/or control algorithms, system and patient state alarms and indicators. These applications may be external to the DiAs system, and may be developed by third parties. Such applications may use DiAs services provided that they comply with the data exchange standards of the system. For example, a Hyperglycemia Mitigation Ser-vice (HMS) is a closed-loop control algorithm or method included in one of the embodiments of DiAs;

The user interface with the system can be custom designed to meet the needs of specific implementations.

Several system/patient status inquiry icons open additional interfaces allowing the patient to access graphical and numerical representation of his/her glucose control, or inform the system of events (such as carbohydrate intake or exercise), which are treated as additional inputs by the analytical system;

Network service (described in the next section) ensures remote monitoring and transmission of alerts and critical information in high-risk states.

In another aspect the invention provides a controller for an artificial pancreas that automatically directs the delivery of insulin to maintain blood glucose concentrations of people with type 1 diabetes mellitus (T1DM) within the euglycemic zone (80-140 mg/dL) using a control algorithm that continuously modulates the control objective depending on the time of day, wherein target blood glucose zone values during the night are higher than during the day, there is a period of smooth transition between the daytime and night-time, and insulin input constraints enforced by the controller are lower during the night than during the day, wherein elevated blood glucose levels are permitted during the night, and the maximum amount of insulin deliverable by the artificial pancreas device while the patient is asleep is reduced, and risk of controller induced hypoglycemia during periods of sleep is thus reduced. In another aspect the invention may provide a periodic zone model predictive control (PZMPC) controller adapted for directing insulin, particularly from an artificial pancreas. The invention also provides corresponding algorithms for programming the subject controllers to effectively implement the disclosed control steps.

The invention also provides an artificial pancreas system or subsystem comprising a subject controller, which may comprise for example, the controller and a pump (e.g. subcutaneous). The invention also provides a periodic zone model predictive control (PZMPC) scheme of an artificial pancreas (AP) for Type 1 diabetes applications comprising a control algorithm which directs a subject controller. The invention also provides a method comprising directing and optionally, delivering, insulin delivery using a subject controller.

The invention includes controllers, algorithms and insulin directing systems essentially as described herein, and all combinations of the recited particular embodiments. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto with-out departing from the spirit or scope of the appended claims.

Software/Hardware Implementation: Central to DiAs is a scalable software stack with a modular design that can be efficiently adapted to a variety of hardware plat-forms. The software architecture, the availability of suitable hardware platforms and opportunities to trans-fer software modules to commercial partners will factor into the choice of DiAs operating systems. For clinical trials and ambulatory implementation, hardware is needed that is portable, rugged, reliable, inexpensive present invention. For example, a method or system of an embodiment of the present invention may be implemented using hardware, software or a combination thereof and may be implemented in one or more computer systems or other processing systems, such as personal digit assistants (PDAs) or cell phone application(s) equipped with adequate memory and processing capabilities. In an example embodiment, the invention was implemented in software running on a general purpose computer.

The computer system may include one or more processors, such as processor. The processor is connected to a communication infrastructure (e.g., a communications bus, cross-over bar, or network). The computer system may include a display interface that forwards graphics, text, and/or other data from the communication infrastructure. In this regard, a cell phone or a tablet computer could be selected. Consequently, the system may run within a customized version of the Android operating system. Android has a robust development environment, is available with source code, is backed by Google and runs on an ever-increasing array of cell phones and tablets from a variety of manufacturers. Android is being adopted by many commercial developers for new embedded soft-ware projects. Although many current products with embedded control software either have no operating system at all or use a simple control loop the trend is towards basing new embedded software projects on Android and embedded Linux. Since Android is built on top of Linux, an Android-based operating system for buffer not shown) for display on the display unit. Display unit may be digital using iOS or Android or another operating system, and/or may be analog.

The computer system may also include a main memory, preferably random access memory (RAM), and may also include a secondary memory. The secondary memory may include, for example, a hard disk drive and/or a removable storage drive, representing a floppy disk drive, a magnetic tape drive, an optical disk drive, a flash memory, etc. The removable storage drive reads from and/or writes to a removable storage unit in a well-known manner. It may use a mobile data storage such as a flash drive or cloud connection, which is read by and written to by removable storage drive. As will be appreciated, the removable storage unit includes a computer usable storage medium having stored therein.

The program would allow transfer of software code to industry partners for commercial use. Android also provides a rich software development kit that supports multi-touch graphical user interface design, data communications, geo-location and telephony.

A diabetic patient is himself the best resource available to manage his diabetes. Meals, insulin dosing, and changes in personal well-being, as well as departures from such plans, are best known to the patient, who alone is ultimately responsible for adherence to a management regimen. The method is performed as a series of process steps or operations executed by one or more patient-operable devices, including personal computers, personal digital assistants, programmable mobile telephones, intelligent digital media players, or other programmable devices, that are working individually or collaboratively to apply a personal predictive management tool.

Modeling involves projecting the glycemic effect of planned meals in light of insulin dosing, as well as any other medications, if applicable. Meal planning is particularly important, where the content and timing of meals greatly impacts blood glucose and must be closely controlled by dosed insulin to compensate for the lack of naturally-produced insulin. The management tool performs dietary planning, which involves determining the glycemic effect of food based on a standardized meal. In a further embodiment, planning also includes projecting the effect of exercise or physical activities that are likely to require appreciable caloric expenditure. Other planning aspects are possible.

Once each planned meal is known, the management tool can model the time courses and amplitudes of change for the meal, dosed insulin, and other non-insulin medications. Additionally, the management tool can be calibrated as necessary to adjust for self-testing and data recorded by the patient through predictive modeling and calibration Personalized models of blood glucose affect for insulin time course, the time courses of other medications, and digestive response are established. The models predict the timing and rise or fall of the patient's blood glucose in response to insulin, other medications, and food. Other modeling and calibrations are possible.

Despite many decades of experience, blood glucose management still involves an educated guess at proper insulin dosing, as the content and timing of meals, dosing and timing of insulin, and patient-specific sensitivities can cause departure from expected blood glucose control directions. For instance, the digestive response of each patient's body to food consumption is unique. However, the digestive response characteristics can be normalized through consumption of a standardized test meal, such as a specific number of oat wafers, manufactured, for instance, by Ceapro Inc., Edmonton, Canada, or similar calibrated consumable. It may be calibrated using a test curve. The test meal contains a known quantity of carbohydrate with a specific glycemic index. Thus, the curve can be adapted to other types of foods to estimate glycemic effect and counteraction by insulin dosing. Other models of digestive response are possible.

### Using Patient Data to Predict Glycemic Events

Some embodiments focus on analyzing historical patient data to predict glycemic events and react quickly in the case of hyperglycemia. They rely on models of insulin and other diabetes medications and how these affect the human body, though some allow for prediction based on an individual's data. Some can calculate the amount of carbohydrates the patient needs in order to recover without overcorrecting.

Some embodiments may include a system and method for Managing Type 1 Diabetes that is patient specific and can be provided for any use at any time. The system provides models on glycemic effect by insulin, antidiabetic and oral medications, and food consumption based on sensitivities to a particular patient and is adjusted based on carbohydrate sensitivity. The amount of insulin necessary to counteract the rise of blood glucose is determined. It also estimated rise in blood glucose. Some embodiments may include a personal prediction tool that approximates what the pancreas does over time to prevent glycemic episodes. Instructions with visuals may be included for the patient and/or caregiver.

Some embodiments may include a portable device that assists in the personalized treatment of diabetes. The invention has a reprogrammable treatment algorithm, an instrument for measuring blood-glucose level, an input device for data, a dosage determinator, an individualized algorithm, a communications port, and a portable package. The invention uses conventional disposable test strips to measure blood glucose. The internal database holds carbohydrate content of common foods. It also has a single enclosure that holds all components of the device. The device's algorithm can be personalized by the particular patient's healthcare professional.

Minerva will use existing database data to provide a basic model, and a treatment plan can then be customized based on the individual's collected data. Minerva, however, has more of an emphasis on individualized treatment and predicting glycemic events based on that specific patient's history, since T1D can behave differently, requiring different treatment depending on the individual, their age, their weight, etc. Minerva's use of CGM is also an improvement over traditional test-strip testing used in older patented technologies, and also will have some ability to take events into consideration when evaluating risk (ex. poker game) and notify the patient appropriately.

The present invention connects the patient's CGM sensor 301 and other sensors 302 to the network 305 and then to their insulin pump 303. The network may also be able to communicate with the insulin pump and take input and instructions from parents and/or caregivers and relay this to the pump. The present invention then takes patient historical data 304 and database data and machine learning algorithm 306 to find predictive patterns 307 in the patient's CGM which may help it prevent if a patient's blood sugar is about to go low or high. This information is relayed to the insulin pump, which can use this information to detect and react to these patterns to predict and prevent glycemic events. If the insulin pump detects a pattern indicative of a future glycemic event 309 310 it can recommend or administer treatment appropriately. If no pattern is found 312 the pump will continue monitoring and analyzing as normal 313.

Some embodiments may compare the individual's glycemic events and data to that of other diabetics to look for irregularities and indicators of other health issues. Other embodiments may allow the program to vibrate the pump loudly in the instance of a glycemic event. If the patient is unresponsive, the app can contact caregivers and/or emergency authorities. Some embodiments may prioritize age in their calculations, and may choose to take less risk with small children who are more prone to glycemic events than adults and teens because of their size.

Minerva has done a series of analyses that have allowed us to retrofit a curve predicting lispro insulin action over time. It correlates closely with published pharmacologic curves but, interestingly, varies in peak timing. That insulin action curve is critical in determining CGM predictions. Our method can individualize that curve for anyone using our system. We are currently working on a predictive CGM curve using that insulin action curve, our calculated ISF and other factors. Some proposed factors are derivatives of straightforward data. Some are contextual, such as time of day, relationship to new pump site and time after a meal. Our plan is to use machine learning to hone this predictive curve so that it can predict lows in sufficient time for preventive action to be taken. In addition, it can be used to predict out the consequences of insulin corrections for high CGM values. Minerva Diamon may use any of the following to predict glycemic events, and there may also be additional factors:

ISF values: Minerva has seen a clear 24-hour pattern to ISF values. Here are some questions we might explore in this regard—some of these we've already coded into the database. Many would be new but presumably suitable to the new dataset. Then, Minerva will analyze:

Does this hold for the group as a whole?

Are there reliable patterns within each individual?

Can we match those patterns and then look at the demographic characteristics of the matching group?

How do we match those patterns: by time of day, by time after a meal, are they seasonal?

Do they have more to do with age or with time after diagnosis?

I:C Ratios: Minerva has seen that insulin to carb ratios seem to be consistent over periods of weeks and then can have abrupt changes that are either situational or seasonal. As a prime example: September and back to school seem to bring a big change in I:C ratios—suddenly the same regimen leads to lows. Conversations with nurse educators confirm this phenomenon as a frequent problem. Again, similar questions as for ISF values can be asked?

What is the consistency of I:C ratios over a longitudinal period?

Are there similarities by age (possibly a weight factor?), by time after T1D? Are there any groupings that follow the same pattern?

Are they seasonal? And, if so, do the “seasons” have to do with general national schedules like school, Christmas vacations, summer vacations?

We are in the process of evaluating how best to statistically evaluate the factors that we theorize are possible markers of an impending low. We know that a mismatch of insulin to carbs causes lows, but that relationship changes depending on contextual variables. For example, exercise has an effect as does emotional stress.

Ideally, we will find markers well enough ahead of time that we can turn off the pump. Or if the predictive factor is a certain ratio of insulin to carb, or an absolute value of the preceding bolus, then we could pre-empt by advising to not give that insulin from the start.

As above, the same questions need to asked of existing database data.

Do we see patterns for the group that would speak to a generalizability of predictive factors? Or at least predictive markers?

Are there patterns for a subgroup that can be identified well enough to plug that factor into a predictive algorithm?

Can we develop a mathematical or statistical approach that will personalize the predictive algorithm? Patient A's CGM may drop at a different rate from Patient B's before a low. Or the ratio of active insulin to carbs on board may differ between patients. Can we teach the computer to rewrite the algorithm for an individual? How many lows do we need to create a good algorithm for that individual? Can we use a continuum to create the algorithm or do we need “low events”?

Patient logs document the interaction of food, other medications, if applicable, and patient sensitivities. However, descriptions of food consumed and manner of preparation, precise times between insulin dosing and completion of a meal, and physiological factors, such as mood or wellness, are generally omitted. Further, physician review normally only occurs during clinic visits, or as necessary, but detailed study is infrequent due to the time, effort, and cost of reviewing every Type 1 diabetic patient.

The accuracy and timeliness of a Type 1 diabetes management regimen can be improved by automating day-to-day managerial aspects, which are historically performed through intuition and sporadic re-evaluation. Automation is introduced to move the management cycle significantly closer to a closed loop arrangement. Control is streamlined and steps that remain to be performed by a patient manually are minimized, or even eliminated, which makes such steps less apt to be forgotten or missed.

An automated diabetes management tool applies heuristics to model and calibrate a personalized diabetes control regimen, as further described below beginning with reference. Through the management tool, the patient can plan and time insulin dosing and meals more accurately than possible through conventional means, including exchange lists and carbohydrate counting. Dynamically tracked blood glucose predictions can also be provided, and self-testing results can be provided directly into the management tool for time-responsive integration and evaluation. In a further embodiment, emergent glucose self-testing, such as interstitial glucose testing, supplements manual self-testing or, where sufficiently reliable, replaces manual self-testing to achieve a fully closed loop system when combined with insulin pump therapy. Other management tool aspects are possible.

Personalized Type 1 diabetes mellitus management can be provided through a patient-operable interface through which glycemic effect prediction and patient interaction can be performed. The user interface provides logical controls that accept patient inputs and display elements that present information to the patient. The logical controls include buttons, or other forms of option selection, to access further screens and menus to estimate glucose rise and insulin needed to counteract the rise because of food, specify an insulin bolus dosage, specify other antidiabetic and oral medications, enter a measured blood glucose reading; and edit information (“EDIT”). Further logical control and display elements are possible.

To assist the patient with planning, a graphical display provides a forecast curve 67, which predicts combined insulin dosing, antidiabetic and oral medication administration, and postprandial blood glucose. Modeling estimates the timing and amplitude of change in the patient's blood glucose in response to the introduction of a substance, whether food, physiological state, or drug, that triggers a physiological effect in blood glucose. Generally, actions, such as insulin dosing, medication administration, exercise, and food consumption cause a measurable physiological effect, although other substances and events can influence blood glucose. The time courses and amplitudes of change are adjusted, as appropriate, to compensate for patient-specific factors, such as level of sensitivity or resistance to insulin, insulin secretion impairment, carbohydrate sensitivity, and physiological reaction to medications. In a further embodiment, the management tool includes a forecaster that can identify a point at which an expected blood glucose level from the personal insulin response profile is expected to either exceed or fall below a blood glucose level threshold, which respectively corresponds to hypoglycemia and hyperglycemia. Other actions and patient-specific factors, like exercise or supervening illness, may also alter the time courses and amplitudes of blood glucose.

Estimating postprandial glucose rise involves modeling food constituents as combined into a meal of specific food types, portion sizes, and preparations. Dietary management, and thence, the management tool, focuses on carbohydrates, which have the greatest effect on blood glucose. Simple sugars increase blood glucose rapidly, while complex carbohydrates, such as whole grain bread, increase blood glucose more slowly due to the time necessary to break down constituent components. Fats, whether triglyceride or cholesterol, neither raise nor lower blood glucose, but can have an indirect affect by delaying glucose uptake. Proteins are broken down into amino acids, which are then converted into glucose that will raise blood glucose levels slowly. Proteins will not generally cause a rapid blood glucose to rise. Nevertheless, both fats and proteins are incorporated into the model by virtue of their empiric effect on blood glucose levels. Additionally, various combinations or preparations of medications or food can have synergistic effects that can alter blood glucose rise and timing.

In the management tool, different meal combinations can be composed by selecting individual foods from a food data library, which stores glycemic effect, digestive speeds and amplitudes as a function of carbohydrate content. The food data library is displayed as food choices. For convenience, portion size and preparation, where applicable, are included with each food choice, although portion size and preparation could alternatively be separately specified.

The food choices are open-ended, and one or more food items may be added to a planned meal by pressing an add item button. Glycemic effect data, such as the glycemic
index and carbohydrates type and content fora particular food item, are retrieved also from the stored food data library and displayed. A cumulative digestive response curve 75 is generated. The digestive response curve estimates digestive speed and amplitude for the individual patient, which traces postprandial blood glucose rise, peak, and fall based on the patient's carbohydrate sensitivity.

A planned meal must be evaluated to determine the insulin needed to compensate for the estimated postprandial rise in blood glucose. In general, food consumption modeling focuses on carbohydrates. Simple sugars, the most basic form of carbo-hydrate, increase blood glucose rapidly. Conversely, more complex carbohydrates, such as whole grain bread, increase blood glucose more slowly due to the time necessary to breakdown constituent components. Proteins also raise blood glucose slowly, as they must first be broken down into amino acids before being converted into glucose. Fats, which include triglycerides and cholesterol, delay glucose uptake. Thus, carbohydrates, and not proteins or fats, have the largest and most direct effect on blood glucose. Notwithstanding, all foods that contribute to blood glucose rise, not just carbohydrates, can be included in the model.

Each item of food consumed contributes to the over-all carbohydrate content and, thence, postprandial blood glucose rise. The type of food and manner of preparation can affect glucose uptake. Orange juice is a beverage that is readily metabolized and absorbed into the blood stream, which results in a rapid and significant rise in blood glucose. The rise, though, is short term. In contrast, steak is primarily protein and the manner of preparation will have little effect on carbohydrate content. The rise in blood glucose is delayed by the protein having to first be broken down into amino acids. The resultant equivalent carbohydrate content also is low, thus resulting in a more attenuated rise in blood glucose. Food items principally containing complex carbohydrates are more affected by manner of preparation. For example, pasta pre-pared “al dente” is slightly undercooked to render the pasta firm, yet not hard, to the bite. The “al dente” form of preparation can increase digestive time and delay glucose uptake. The form of preparation can also be taken into account in the management tool. Finally, some medications can modify the effect of foods on blood glucose. Other effects on food items, as to type and manner of preparation, also are possible.

Except for the occasional snack item, food is generally consumed as a meal. Items of food consumed in combination during a single sitting, as typical in a meal, can cumulatively or synergistically interact to alter the timing and amplitude of blood glucose rise based on the digestive processes involved and the net change to overall carbohydrate content. The cumulative digestive response curve combines the respective constituent digestive response curves and proportionately applies the patient's carbohydrate sensitivity. The cumulative curve has an initial near-term peak, which reflects the short time course and high glucose content of the orange juice, and a delayed long term peak, which reflects the protein-delayed and significantly less-dramatic rise in blood glucose attributable to the sirloin steak. Shortly following consumption of the orange juice and steak, blood glucose rise is dominated by the effects of the orange juice while the steak has little effect. Later, the effects of the orange juice dwindle and the effects of the steak dominate the rise in blood glucose. The effects of both foods are present in-between.

Both the types of available food items and their accompanying data may change over time. At a minimum, the food data library contains glycemic effect, digestive speeds and amplitudes as a function of carbohydrate content. The data can be obtained from various sources and is integrated into the library. For instance, standardized carbohydrate values 122, for instance, glycemic indices or glycemic load, can be retrieved from authoritative sources, such as the University of Toronto, Toronto, Ontario, Canada. Empirical values can be derived by the patient through experiential observations of glycemic effect by a combination of fasting and pre- and postprandial blood glucose testing. Synergistic values of food combinations, perhaps unique to the patient's personal culinary tastes, could similarly be empirically derived. Other food data values 125 and sources of information are possible.

In the course of providing blood glucose management, a more proactive approach can be taken as circumstances provide. Interaction refers to the undertaking of some action directly with or on behalf of the patient. The interaction can include suggesting opportune times to the patient at which to perform self-testing of blood glucose or to pay attention to the values in case of individuals using CGM. Such times include both pre- and postprandial times, particularly when blood glucose rise is estimated to peak. Similarly, alerts can be generated), for example, warnings of low blood glucose, or reminders provided such as reminding the patient to take his insulin for high glucose levels. Interaction could also include intervening (operation 136), such as notifying a patient's physician or emergency response personnel when a medical emergency arises. Other forms of patient interaction are possible.

### Facilitating Communication Between Patient and Caregiver

Prior art makes patient information available via the internet or a paired embodiment, and sometimes allows a caregiver to send instructions to the patient. Some prior art can track changes in the patient, comparing their data against that of others with diabetes to look for abnormalities.

The present invention connects the patient's CGM sensor 301 and other sensors 302 to the network 305 and then to their insulin pump 303. The network then makes the patient's data available through a mobile device with app 405, a web portal 406, and/or a smart watch 407. Any of these three are capable of connecting with both a primary caregiver 408 and/or one or more secondary caregivers 409. The program also facilitates communication between the program and its own machine learning-based instructions and calculations, as well as facilitate communication 410 between the primary 408 and secondary 409 caregiver(s). It may also be possible to add multiple primary caregivers and facilitate communication between them, such as in the instance of a child with two or more parental/relative caregivers.

Some embodiments may have the ability to add users (babysitters, etc.) to an app that facilitates communication between them and the patient's primary caregiver and recommends treatment, send notifications to caregiver if there is an abnormal response to insulin, or if the patient is unresponsive for a set period of time. Other embodiments may automatically connect patient data to doctors, insurance, and/or other healthcare providers.

It may be possible to extend the present invention to replace the DMMP (Diabetes Medical Management Plan), used in schools to manage the condition of children with diabetes. The DMMP is a form required by each state for all students with diabetes. It is filled out by a physician caring for such student and lists instructions for a school nurse to follow. Each state can have its own version but they are largely the same and there is a national prototype (attached). Minerva could extend the Diamon app to act as an electronic version of the DMMP. It would be a relatively smooth extension of the current Diamon app. The plan includes:
Create an online DMMP form.

Incorporate Diamon instructions into that form so that, once the form is filled, those instructions flow into the Diamon School Mode.

The “administrator” for that Mode would be exclusively the healthcare team, essentially giving an electronic signature to the Diamon DMMP and the Diamon School Mode. Any changes to those instructions could only be filled by the healthcare provider. Changes could be implemented any time online.

The Menu page of the Diamon App would include a full version of the DMMP for easy reference by the school nurse. Likewise, the school nurse could have online access to the DMMP of any child using Diamon with the DMMP option.

Minerva would work with the American Diabetes Association “Safe at School” committee to assist in the design and distribution of the Diamon digital DMMP.

In one aspect of the disclosure, a diabetes management system having a reliable data management scheme is disclosed herein. The diabetes management system includes a plurality of devices, each device performing a different function relating to treatment of diabetes of a patient and having a device identifier that identifies a type of the device. Each device generates data records relating to the function of the device, and each device includes a metadata generator con-figured to generate a metadata tag for a data record generated by the device. The metadata tag includes the device identifier of the corresponding device, a record identifier, and a source identifier indicating whether the record was originated by a human or a device. The system further comprises a diabetes management device in electronic communication with the plurality of devices, wherein the diabetes management device is configured to manage records received from the plurality of devices. When a first device of the plurality of devices generates a new record to be communicated to the diabetes management device, the metadata generator of the first device generates a new unique record identifier and a new metadata tag based on the new unique record identifier and the device identifier of the first device, and the first device propagates the new record and the new metadata tag to the second device.

During a healthcare consultation, the patient typically shares with the clinician a variety of patient data including blood glucose measurements, continuous glucose monitor data, amounts of insulin infused, amounts of food and beverages consumed, exercise schedules, and other lifestyle information. The clinician may obtain additional patient data that includes measurements of HbAlC, cholesterol levels, triglycerides, blood pressure, and weight of the patient The patient data can be recorded manually or electronically on a handheld diabetes management device, a diabetes analysis software executed on a personal computer (PC), and/or a web-based diabetes analysis site (not shown). The clinician can analyze the patient data manually or electronically using the diabetes analysis software and/or the web-based diabetes analysis site. After analyzing the patient data and reviewing adherence of the patient to previously prescribed therapy, the clinician can decide whether to modify the therapy for the patient.

Information stored in a specific patient database may be associated with a particular patient by a patient identification number. Similarly, each patient-specific meter can include a meter identification number, and can be associated with its owner via the owner's PIN. When a patient-specific meter uploads data to the patient database, the server may query the meter for a specific PIN and/or MIN in order to ensure that the data being uploaded is stored in the correct patient database 50, and that each measurement is associated with the meter which took it. PINs and MINs can be any suitable alphanumeric, symbolic, binary or other identifier which is sufficiently unique to specifically identify a single patient or meter.

The server can comprise any suitable server hardware, including a purpose-built server system, a personal computer, a storage array, or any combination of suitable hardware components. The server may also include a user interface for allowing a sufficiently authorized user to easily access information contained in the server. A user interface may, in one embodiment, comprise a secure web site accessible by a user via a typical internet connection. Alternatively, a server user interface can comprise a self-supporting communication and connection system. In an alternative embodiment, the server can be omitted from the system, and the storage, processing and communications functions of the server can be implemented in other system components, such as the patient meter itself.

The caregiver, insurance-provider and secondary caregiver terminals may comprise any suitable hardware or information appliance, including but not limited to a personal computer, a “dumb” terminal, a personal digital assistant, phone, pager, two-way pager, interactive television device, telephonic audio interface, etc. Server can provide information to healthcare professionals. Such healthcare professionals can communicate with server via ACC healthcare professionals soft-ware. The server can send healthcare professionals clinical patient data, including test results, time of day, and other information. The server can send healthcare professionals a message when a patient has received a health alert from the ACC system. The server can analyze the data collected from all of a healthcare professional's patients who use ACC system and send information based on the data analysis to healthcare professionals. Such information can include recommendations for how healthcare professionals can improve the care available to their overall patient population. The healthcare professionals' software can include features to make patient data sortable and presentable according to any of a variety of categories of data (e.g., day of test, answer to specific questions, time of test, etc.).

The server can send healthcare professionals various messages. Such messages can include coaching tips and explanations of individual patient data and blood glucose measurements for the healthcare professional to convey to the patient's diabetes management application. Such messages can include other information for healthcare professionals to convey to patients. In some instances, server can provide a healthcare professional tailored advertisements and coupons. The server can inform healthcare professionals of trends related to total patient population care and related tips on caring for the healthcare professional's overall patient population. In some instances, server 40 can provide alerts on which patients need additional care, along with printouts to aid in appointments and research study analysis.

As shown, server can provide information to caregivers (e.g., through ACC caregivers software). Such information can include coaching tips and messages for the caregiver to relay to the patient. In some instances, the caregiver can relay messages to the patient via direct messages that are viewable in the patient's diabetes management application.

The server can provide information to payers (e.g., via ACC payers software).

The server can be configured to analyze overall patient data to generate subscriber population trends and analysis and can send such information to the payer. In some instances, server can compute comparisons of patient populations by clinic or by healthcare provider and can send such comparison information to the payer. In some instances, server can send messages to the payer related to research study analysis.

As shown, server can provide information to various business entities. For example, an administrator or other business can access information from the server to conduct market analysis. An administrator or other business can generate and deliver prompts to patients (e.g., supplies based on tests completed by the patients). In some instances, an ACC administrator or other business may be interested in learning the level of reward and/or the health success level of patients by population. In some instances, the ACC server can provide information.

The server can provide information to government entities. The server can be configured to analyze overall patient data to generate subscriber population trends and analysis and can send such information to a government entity. In some embodiments, server can be configured to compute comparisons of patient populations by clinic or by healthcare provider and can send such comparison information to a government entity. In some instances, server can send messages to the payer related to research study analysis.

The diabetes management application software can allow in-app/in-software messaging between the patient and other users. The server can enable the patient's selected healthcare providers, caregivers, and others (e.g., loved ones) to send messages directly to the patient via the diabetes management application (and vice versa).

In the course of providing blood glucose management, a more proactive approach can be taken as circumstances provide. Interaction refers to the undertaking of some action directly with or on behalf of the patient. The interaction can include suggesting opportune times to the patient at which to perform self-testing of blood glucose (operation 132). Such times include both pre- and postprandial times, particularly when blood glucose rise is estimated to peak. Similarly, alerts can be generated, for example, warnings of low blood glucose, or reminders provided such as reminding the patient to take his insulin for high glucose levels. Interaction could also include intervening such as notifying a patient's physician or emergency response personnel when a medical emergency arises.

The method may further include receiving one of the intervention insulin quantity glucagon quantity, and the intervention carbohydrate quantity. The method may further include entering one of the intervention insulin quantity and the intervention carbohydrate quantity into a database in response to user selection of either of the first and second user intervention mechanisms. The method may further include date and time stamping the one of the intervention insulin quantity and the intervention carbohydrate quantity prior to entry into the database. Some embodiments may include a pump that delivers both insulin and glucagon or a glucagon patch that could be controlled remotely for resuscitation from a low insulin or glucagon level. The ability to use glucagon levels may be added to our algorithms.

The method may further include waiting for a delay time after the user selection of the first user intervention mechanism and prior to including the one of the intervention insulin quantity and the intervention carbohydrate quantity in the execution of the insulin delivery algorithm. A system providing for user intervention in a medical control arrangement may comprise a first user intervention mechanism responsive to user selection thereof to produce a first user intervention signal, a second user intervention mechanism responsive to user selection thereof to produce a second user intervention signal, and a processor executing a drug delivery algorithm forming part of the medical control arrangement. The processor may be responsive to the first user intervention signal to include an intervention drug quantity in the execution of the drug delivery algorithm. The processor may be responsive to the second user intervention signal to exclude the intervention drug quantity from the execution of the drug delivery algorithm including drugs that manage insulin, glucagon, and other levels.

The system may further include means for receiving the intervention drug quantity. The medical control arrangement may be a diabetes control arrangement, the drug delivery algorithm may be an insulin delivery algorithm, and the intervention drug quantity may be an intervention insulin quantity. The processor may be responsive to the first user intervention signal to include the intervention insulin quantity in the execution of the insulin delivery algorithm by adding the intervention insulin quantity to a current insulin bolus amount. The processor may further be responsive to the first user intervention signal to command administration of the combination of the intervention insulin quantity and the current insulin bolus amount to the user.

The system may further include a database having drug delivery information stored therein. The processor may be responsive to either of the first and second user intervention signals to enter the intervention drug quantity into the database. The processor may be configured to date and time stamp the intervention drug quantity prior to entry into the database.

The processor may be operable to wait for a delay time prior to including the intervention drug quantity in the execution of the insulin delivery algorithm.

The processor may be configured to continue uninterrupted execution of the insulin delivery algorithm regard-less of whether the first or second user intervention signal is produced.

A method of allowing user intervention in a medical control arrangement may comprise executing a drug delivery algorithm forming part of the medical control arrangement, monitoring first and second user intervention mechanisms, including an intervention drug quantity in the execution of the drug delivery algorithm in response to user selection of the first user intervention mechanism, and excluding the intervention drug quantity from the execution of the drug delivery algorithm in response to user selection of the second user intervention mechanism.

The method may further include receiving the intervention drug quantity. The method may further include entering the intervention drug quantity into a database in response to user selection of either of the first and second user intervention mechanisms. The method may further include date and timestamping the intervention drug quantity prior to entry into the database.

The method may further include waiting for a delay time after the user selection of the first user intervention mechanism and prior to including the intervention drug quantity in the execution of the drug delivery algorithm.

The medical control arrangement may be a diabetes control arrangement, the drug delivery algorithm may be an insulin delivery algorithm, glucagon delivery algorithm, or other hormone level algorithm, and the intervention drug quantity may be an insulin intervention quantity.

A system providing for user intervention in a medical control arrangement may comprise a first user intervention mechanism responsive to user selection thereof to produce a first user intervention signal, a second user intervention mechanism responsive to user selection thereof to produce a second user intervention signal, and a process or executing a drug delivery algorithm forming part of the medical control arrangement. The processor may be responsive to the first user intervention signal to include an intervention therapy value in the execution of the drug delivery algorithm. The processor may be responsive to the second user intervention signal to exclude the intervention therapy value from the execution of the drug delivery algorithm.

The system may further include means for receiving the intervention therapy value. The input device may be used in a conventional manner to input and/or modify data. In the illustrated embodiment, the display is also included for viewing information relating to operation of the device and/or system. Such a display may be a conventional display device including for example, but not limited to, a light emitting diode (LED) display, a liquid crystal display, a cathode ray tube (CRT) display, or the like. Alternatively or additionally, the display may be or include an audible display configured to communicate information to a user or third party via one or more coded patterns, vibrations, synthesized voice responses, or the like. Alternatively or additionally, the display may be or include one or more tactile indicators configured to display tactile information that may be discerned by the user or a third party.

In one embodiment, the input device may be or include a conventional keyboard or key pad for entering alphanumeric data into the processor. Such a keyboard or key pad may include one or more keys or buttons configured with one or more tactile indicators to allow users with poor eyesight to find and select an appropriate one or more of the keys, and/or to allow users to find and select an appropriate one or more of the keys in poor lighting conditions. Alter-natively or additionally, the input device may be or include a conventional mouse or other conventional point and click device for selecting information presented on the display. Alternatively or additionally, the input device may include the display configured as a graphical user interface (GUI). In this embodiment, the display may include one or more selectable inputs that a user may select by touching an appropriate portion of the display using an appropriate implement. Alternatively or additionally, the input device may include a number of switches or buttons that may be activated by a user to select corresponding operational features of the device and/or system. Alternatively or additionally, the input device may be or include voice activated circuitry responsive to voice commands to provide corresponding input data to the processor

In any case, the input device and/or display may be included with or separate from the electronic device.

In some embodiments, the system may include a number of medical devices. In such embodiments, any of the one or more medical devices may be implanted within the user's body, coupled externally to the user's body (e.g., such as an infusion pump), or separate from the user's body. Alternatively or additionally, one or more of the medical devices may be mounted to and/or form part of the electronic device. The number of medical devices are each configured to communicate wirelessly with the communication I/O unit of the electronic device via one of a corresponding number of wireless communication links. The wireless communications may be one-way or two-way. The form of wireless communication used may include, but should not be limited to, radio frequency (RF) communication, infrared (IR) communication, RFID (inductive coupling) communication, acoustic communication, capacitive signaling (through a conductive body), galvanic signaling (through a conductive body), or the like. In any such case, the electronic device and each of the number of medical devices include conventional circuitry for conducting such wireless communications circuit may further include, as appropriate. Alternatively or additionally, one or more of the medical devices may be configured to communicate with the electronic device via one or more conventional hardwire connections therebetween. Each of the one or more medical devices may include any one or more of a conventional processing unit, conventional input/output circuitry and/or devices and one or more suitable data and/or program storage devices.

The system is, or forms part of, a conventional closed-loop or semi closed-loop diabetes control arrangement. In this regard, the system includes a delivery mechanism for delivering controlled amounts of a drug; e.g., insulin, glucagon, incretin, or the like, and/or offering an alternatively actionable recommendation to the user via the display e.g., ingesting carbohydrates, exercising, etc. The system may be provided in any of a variety of conventional configurations, and examples of some such configurations will now be described. It will be understood, however, that the following examples are provided merely for illustrative purposes, and should not be considered limiting in any way. Those skilled in the art may recognize other possible implementations of a closed-loop or semi-closed loop diabetes control arrangement, and any such other implementations are contemplated by this dis-closure.

In a first example implementation of the system, the electronic device is provided in the form of a conventional insulin pump configured to be worn externally to the user's body and also configured to controllably deliver insulin to the user's body. In this example, the number of medical devices may include one or more implanted sensors and/or sensor techniques for providing information relating to the physiological condition of the user. Examples of such implanted sensors may include, but should not be limited to, a glucose sensor, a body temperature sensor, a blood pressure sensor, a heart rate sensor, or the like. In implementations that include an implanted glucose sensor, the system may be a fully closed-loop system operable in a conventional manner to automatically monitor blood glucose and deliver insulin, as appropriate, to maintain blood glucose at desired levels. The number of medical devices may alternatively or additionally include one or m re sensors or sensing systems that are external to the user's body and/or sensor techniques for providing information relating to the physiological condition of the user. Examples of such sensors or sensing systems may include, but should not be limited to, a glucose strip sensor/meter, a body temperature sensor, a blood pressure sensor, a heart rate sensor, or the like. In implementations that include an external glucose sensor, the system may be a semi closed-loop system operable in a conventional manner to deliver insulin, as appropriate, based on glucose information provided thereto by the user. Information provided by any such sensors and/or senor techniques may be communicated to the system using any one or more conventional wired or wireless communication technique.

In a second example implementation of the system, the electronic device is provided in the form of a handheld remote device, such as a PDA, phone with application, or other handheld device. In this example, the number of medical devices include at least one conventional implantable or externally worn drug pump. In one example, an insulin pump may be configured to controllably deliver insulin to the user's body. In this embodiment, the insulin pump is configured to wirelessly transmit information relating to insulin delivery to the handheld device. The handheld device is configured to monitor insulin delivery by the pump, and may further be configured to determine and recommend insulin bolus amounts, carbohydrate intake, exercise, and the like. The system may or may not be configured in this embodiment to provide for transmission of wireless information from the handheld device 12 to the insulin pump.

In an alternate embodiment of this example, the handheld device is configured to control insulin delivery to the user by determining insulin delivery commands and transmitting such commands to the insulin pump. The insulin pump, in turn, is configured to receive the insulin delivery commands from the handheld device, and to deliver insulin to the user according to the commands. The insulin pump, in this embodiment, may or may not further process the insulin pump commands provided by the hand-held unit. In any case, the system will typically be configured in this embodiment to provide for transmission of wireless information from the insulin pump back to the handheld device 12 to thereby allow for monitoring of pump operation. In either embodiment of this example, the system may further include one or more implanted and/or external sensors of the type described in the previous example.

Those skilled in the art will recognize other possible implementations of a closed-loop or semi-closed loop diabetes control arrangement using at least some of the components of the system. For example, the electronic device in one or more of the above examples may be provided in the form of a laptop, notebook, phone, personal computer, or other device configured to communicate with one or more of the medical devices, at least one of which is an insulin pump, to monitor and/or control the delivery of insulin to the user. In some embodiments, the medical device may control delivery of another diabetes drug such as glucagon or another agent. As another example, the system may further include a remote device (not shown) configured to communicate with the electronic device and/or one or more of the medical devices to control and/or monitor insulin delivery to the patient. The remote device may reside in a caregiver's office or other remote location, and communication between the remote device and any component of the system may be accomplished via an intranet, internet (e.g., world-wide-web), cellular, telephone modem, RF, or other communication link. Any one or more conventional internet protocols may be used in such communications. Alternatively or additionally, any conventional mobile content delivery system; e.g., short message system (SMS), or other conventional message schema may be used to provide for communication between devices comprising the system. In any case, any such other implementations are contemplated by this disclosure.

Generally, the concentration of glucose in a person with diabetes changes as a result of one or more external influences such as meals and/or exercise, and may also change resulting from various physiological mechanisms such as stress, menstrual cycle and/or illness. In any of the above examples, the system responds to the measured glucose by determining the appropriate amount of insulin to administer in order to maintain normal blood glucose levels without causing hypoglycemia. In some embodiments, the system is implemented as a discrete system with an appropriate sampling rate, which may be periodic, aperiodic or triggered, although other continuous (analog) systems or hybrid systems may alternatively be implemented as described above.

As one example of a conventional diabetes control system, one or more software algorithms may include a collection of rule sets which use glucose information, insulin delivery information, and/or subject inputs such as meal intake, exercise, stress, illness and/or other physiological properties to provide therapy, etc., to manage the user's glucose level. The rule sets are generally based on observations and clinical practices as well as mathematical models derived through or based on analysis of physiological mechanisms obtained from clinical studies. In the example system, models of insulin pharmacokinetics and pharmacodynamics, glucose pharmacodynamics, meal absorption and exercise responses of individual patients are used to determine the timing and the amount of insulin to be delivered. A learning module may be provided to allow adjustment of the model parameters when the patient's overall performance metric degrades (e.g., adaptive algorithms, using Bayesian estimates, may be implemented). An analysis model may also be incorporated which oversees the learning to accept or reject learning. Adjustments are achieved utilizing heuristics, rules, formulae, minimization of cost function(s) or tables (e.g., gain scheduling).

However, the human metabolism is complex and not fully understood. The solution space of managing glucose in daily life is currently limited. Day to day variability, incorrect or inaccurate input, device failures, physiological changes, exercise, stress, illness, etc. are known to produce changes in a diabetic person's condition. The working assumptions with conventional diabetes control systems are that the various device components are working correctly, and that the methodology or logic or process of determining therapy conforms to assumptions of operation. These assumptions are generally not accurate with actual diabetes control systems, and physical implementations of conventional diabetes control systems will generally encounter failure modes that the system cannot correct. Such failure modes may be detectable by the diabetes control system, while others may be detectable only by the user.

Models within the system typically use an approximation of the subject and device components to determine the therapy. The structures and parameters of the models define the anticipated behavior. However, the assumptions of the models may be inaccurate; the internal states of the models may not match with the actual subject, thereby leading to performance errors.

One example model, and potential sources of performance errors associated therewith, is a meal model. Errors in predicting meal absorption characteristics may result from inaccuracies in the dynamic behavior described by the shape of the user's carbohydrate absorption profile.

Errors in timing as well as in shape of the profile may cause the diabetes control system to drive the user's glucose level toward hyperglycemic or hypoglycemic conditions. Similar considerations and error sources exist with respect to glucose measurement subcutaneous models, insulin absorption subcutaneous models (for various insulin types), exercise models, stress models glucose-insulin dynamics, and the like.

Any of a variety of conventional controller design methodologies, such as PID systems, full state feedback systems with state estimators, output feedback systems, LQG controllers, LQR controllers, eigen value/eigen structure controller systems, and the like, could be used to design algorithms to perform physiological control. They typically function by using information derived from physiological measurements and/or user inputs to determine the appropriate control action to use. While the simpler forms of such controllers use fixed parameters (and therefore rules) for computing the magnitude of control action, the parameters in more sophisticated forms of such controllers may use one or more dynamic parameters. The one or more dynamic parameters could, for example, take the form of one or more continuously or discretely adjustable gain values. Specific rules for adjusting such gains could, for example, be defined either on an individual basis or on the basis of a patient population, and in either case will typically be derived according to one or more mathematical models. Such gains are typically scheduled according to one or more rule sets designed to cover the expected operating ranges in which operation is typically nonlinear and variable, thereby reducing sources of error. Errors in such feedback systems are, however, present, and therefore may accumulate and lead to unacceptable system inaccuracies.

Models describing the patient, for example, can be constructed as a black box wherein equations and parameters have no strict analogs in physiology. Rather, such models may instead be representations that are adequate for the purpose of physiological control. The parameters are typically determined from measurements of physiological parameters such as blood glucose, insulin concentration, and the like, and from physiological inputs such as food intake, alcohol intake, insulin doses, and the like, and also from physiological states such as stress level, exercise intensity and duration, menstrual cycle phase, and the like. These models are used to estimate current glucose or to predict future glucose values. Insulin therapy is derived by the system based on the model's ability to predict glucose for various inputs. Other conventional modeling techniques may be additionally or alternatively used including for example, but not limited to, building models from first principles. Errors in any such model types may result from a variety of causes such as incorrect estimation of model parameters, parameters that are non-linear and/or time varying, unmodeled system dynamics, incorrect dynamics, and the like.

Errors arise from delays in action response, delays in measuring glucose, processing delays, delays caused by system operation cycle step size, and the like. It is also desirable to provide for the ability to recover from situations that the system does not or cannot detect as failures. For example, as a result of one or more of the above-described system error sources, the system may drive the user's insulin sufficiently toward hyperglycemia or hypoglycemia that the user identifies or realizes the resulting symptoms even though the system does not indicate any errors or failure modes. System errors/failures and/or user symptoms may be accelerated or decelerated as a result of the user's physiological state including, for example, illness, stress and the like.

The system provides for user intervention in the diabetes control arrangement of the type or types described hereinabove. In particular, the input device includes one or more user intervention input mechanisms that allow the user to intervene in the controlled insulin delivery algorithm being executed by a diabetes control arrangement in a manner that allows the insulin delivery algorithm to continue executing without resetting or otherwise disabling the algorithm and/or system. By appropriate selection/activation of the one or more user intervention input mechanisms, the user can take corrective action and then either allow the insulin delivery algorithm to act upon the corrective action (optionally with or without a delay) by including the corrective action in the execution of the insulin delivery algorithm, or to disregard, and not act upon, the corrective action by excluding the corrective action in the execution of the insulin delivery algorithm.

In either case, though, the user enters the corrective action into the system. In one embodiment, the input device includes two user-selectable buttons. By pressing one of the two user-selectable buttons, the user can intervene in the diabetes control arrangement, take corrective action and then allow the insulin delivery algorithm being executed to act upon the corrective action. By pressing the other of the two user-selectable buttons, the user can intervene in the diabetes control arrangement and take corrective action with the corrective action being excluded from the insulin delivery algorithm being executed. In either case, the corrective action is entered into the database in the memory unit or other data storage device. Also, in either case the insulin delivery algorithm continues to execute, and may also process the user intervention information depending upon appropriate selection of the user intervention input mechanism.

In an alternate embodiment, the display includes a graphical user interface (GUI) that allows the user to select, at will, either of two user-selectable display icons. Selecting either of the two display icons will, in this embodiment, have the same effect as the selecting either of the two user-selectable buttons in the previous example. It will be understood that more, fewer, and/or other user-selectable input mechanisms may be provided to allow the user to intervene, at will, in the diabetes control arrangement, and to select between allowing the system to act upon the corrective action taken in the intervention and having the system disregard the corrective action taken in the intervention. Any such alterative user-selectable mechanisms are contemplated by this disclosure.

The user may intervene in the diabetes control arrangement, as just described, for the purpose of taking either of two possible corrective actions; namely, taking action to reduce the user's glucose level or taking action to increase the user's glucose level. Conventional mechanisms for reducing the user's glucose level include, but are not limited to, dispensing insulin into the user's body, such as in the form of a bolus and exercising. Conventional mechanisms for increasing the user's glucose level include, but are not limited to, ingesting carbohydrates and dispensing glucagon and possibly glucose into the user's system. Either corrective action taken by the user is independent of the system logic and consideration of devices within the system. Such user intervention allows the system to continue operation under the insulin delivery algorithm while also allowing the system to recover without necessarily requiring a system reset.

The algorithm will typically be stored in the memory unit or other data storage device, and will be executed by the processor. In the illustrated embodiment, it will be understood that the processor will be, simultaneously or in tandem, executing one or more conventional insulin delivery algorithms configured to manage or control delivery of insulin to the user, and that the algorithm will therefore be executed by the processor as an independent algorithm. Alternatively, the algorithm and the one or more conventional insulin delivery algorithms may be executed by different processors in an embodiment of the system that includes multiple processors. In any case the algorithm will be described for purposes of this document as being executed by the processor, which would be in the cloud and may have a backup processor on the local device. In this description, it will be understood that the algorithm treats user interventions as asynchronous occurrences requiring immediate attention, as compared with synchronous, e.g., periodic, events that the system normally manages in accordance with the one or more insulin delivery algorithms.

As described hereinabove, the user may intervene in the diabetes control arrangement, as just described, for the purpose of taking either of two possible corrective actions; either by taking action to decrease the user's glucose level, e.g., by receiving insulin, such as in the form of a bolus, and/or via one or more other conventional glucose decreasing mechanisms, or by taking action to increase the user's glucose level, e.g., by ingesting carbohydrates and/or via one or more other conventional glucose increasing mechanisms. In cases where the user chooses to intervene by taking additional insulin, the user may do so via any conventional technique. Examples include, but are not limited to, manually overriding the system in a conventional manner to direct the system to deliver a specified amount of insulin, programming the system in a conventional manner to deliver the specified amount of insulin, manually injecting the specified amount of insulin via a syringe, or the like.

In any case, the user enters the specified amount of insulin into the system via an appropriate one of the input devices, and the processer receiving the specified amount of insulin, or intervention insulin quantity (IIQ), from the input device. In cases where the user chooses to intervene by ingesting carbohydrates, the user enters the quantity of carbohydrates that were ingested into the system via an appropriate input device. The processor executes step in this case by receiving the intervention carbohydrate quantity (ICQ) from the input device. In either case, it will be understood that the algorithm will also typically include one or more steps providing a timeout mechanism that allows the algorithm to continue execution after a predefined time period when the user fails to enter, or incompletely enters, IIQ or ICQ information at step. Any such one or more steps would be a mechanical exercise for a skilled algorithm designer.

It will be understood that in one or more embodiments of the system, it may be desirable to synchronize date and/or time stamping of IIQ with a reference date and/or time using one or more conventional date and/or time synchronization techniques. It will also be understood that the IIQ data is date and time stamped, and then stored in the memory unit or other data storage device, at or near the time that the intervention insulin quantity, IIQ, is scheduled for delivery, or actually delivered, to the user. In other embodiments, the appropriate time to date and time stamp IIQ and enter this information into the memory unit or other data storage device will become apparent. As one specific example, in embodiments where the intervention insulin quantity, IIQ, is manually administered, it will be appropriate to date and time stamp the IIQ data at or near the time that the intervention insulin quantity is actually administered; e.g., such as directly following step of the algorithm. Similar considerations apply to the date, time stamping and storage of the intervention carbohydrate quantity, ICQ.

The systems and embodiments may also be used to assist medical professionals or treat conditions other than diabetes. Possible uses include Management of hypertension with automated BP cuff input from a Bluetooth or otherwise connected measuring device, management of tachycardias, arrythmias and atrial fibrillation rate control from a pulse or EKG wearable, general anesthesia or intravenous sedation which could be managed via EKG, BP, and pulse oximetry and end-tidal carbon dioxide monitoring all connected to our system. Algorithms could drive the output which would be via Bluetooth or otherwise connected Intravenous pumps with anesthetics such as propofol or fentanyl.

Systems and methods could be used as or in combination with A setup system whereby an administrator chooses parameters by which to drive an algorithm. For hypertension,
e.g. the setup might say: “For BP >140 systolic and <180, change meds to 1.5 tabs Diazide per day. A “fetch” server which gets the physiologic input from another server or directly from devices; a “save” program which places the inputs in an ordered database; an “evaluate” program which compares the inputs to known parameters. These “known” groups of parameters are determined by the particular disease state or process, they will be incorporated into the setup and the individual practitioner can adjust the ranges to be personalized. It may also include an output program that either sends instructions or notifications to the user. In the case of anesthetics, the output could be like an artificial pancreas and drive the pumps themselves.

### Gamification and Education

To motivate users, particularly children and teenage patients, there may be a gamified system designed to encourage them to follow Diamon's recommendations, log meals, report symptoms, etc. There may be a leaderboard which can show anonymized data (such as average CGM data and/or Alc data or other physiological parameters) which can be filtered so that they are more relevant (by age, sex or other parameters) to provide a leaderboard which can provide a competitive framework which provides a means by which the end-users can compare their information and provide a motivation for improving their insulin control. Educational can also be provided by on-demand care provider lectures and/or real-time consultations. This educational component can also include methods to assess and determine what motivates an individual (such as use of particular applications—e.g., Kahoot, Quizlet) along with the personalized recommendations that offer the greatest impact on measured glucose control.

Related to or separate from the gamification component, a dashboard may provide a combination of glucose control (which can be ‘level set or filtered to match a corresponding and relevant peer group of the end-user), food and exercise knowledge as measured from quizzes and other assessments, as well as the leaderboard. The dashboard could also display wearable data, sleep data, stress information, etc. This data can be obtained from wearables as well as in-clinic assessments.

What is claimed is:

1. A method for managing glucose levels in a patient, comprising:
continuously collecting glucose data from the patient;
processing the glucose data; and
using a machine learning algorithm personalized to the patient that uses machine learning for continuously predicting and determining at least one recommended treatment for the patient, wherein
a history of the glucose data and treatments is stored in a database by at least one of the patient and a caregiver, and
the machine learning algorithm uses the history, processed glucose data, and other personalized data to predict and calculate a recommended treatment; and
at least one of the patient and the caregiver is provided with real-time updates of the history on demand via a web portal or mobile device.

2. The method of claim 1, wherein the machine learning algorithm creates a model to predict how the patient will respond to various food consumptions or activities and uses machine learning to modify the model based on the processed glucose data, treatments and personalized data provided by the patient or the caregiver.

3. The method of claim 1, wherein the machine learning algorithm is a cloud-based machine learning algorithm that provides predictive analytics and optimization of therapeutic timing and dose delivery of insulin, naturally occurring mediators, synthetic mediators, and diet recommendations.

4. The method of claim 1, wherein the personalized data includes foods commonly eaten by the patient and the machine learning algorithm instructs treatment based on anticipated consumption of foods commonly eaten by the patient.

5. The method of claim 1, wherein the personalized data includes activities performed by the patient and the machine learning algorithm instructs future treatment based on anticipated activities of the patient.

6. The method of claim 1, wherein the history for the patient are accessible to the patient and the caregiver by accessing the database, and wherein the database is cloud based.

7. The method of claim 1, wherein personalized data is provided by the patient or the caregiver and includes wellness, food and drink consumption, and activities of the patient, and wherein faulty data included in the personalized data is removed by the patient or the caregiver.

8. The method of claim 1, further comprising presenting instructions and educational information to the caregiver to assist the patient in critical situations.

9. The method of claim 1, wherein the machine learning algorithm uses machine learning to enhance accuracy of the predictions and treatments.

10. The method of claim 1, further comprising positively rewarding the patient to encourage the patient to respond quickly to treatment instructions.

11. The method of claim 1, wherein the history is accessible from a mobile device using one of an app, web portal, and smart watch.

12. The method of claim 1, further comprising using an intervention feature to administer necessary drugs to the patient.

13. A system for managing glucose levels in a patient, comprising:
a continuous glucose monitor (CGM) configured to collect glucose data from the patient;
a computer processor operatively connected to the CGM, the computer processor configured to receive and process the glucose data;
a database operatively connected to the computer processor, the database storing the collected glucose data and the processed glucose data; and
a decision tree algorithm being personalized to the patient and stored on the computer processor, the decision tree algorithm configured to predict and determine at least one recommended treatment for the patient based on the processed glucose data by using machine learning and algorithms to identify patterns based on
combined collected and processed glucose data from the patient with collected and processed glucose data of one or more other patients stored in another database, and
personal data of the patient provided by one of the patient, a caregiver, and a professional;
wherein the decision tree algorithm uses machine learning to make corrections or changes in real time to the recommended treatments.

14. The system of claim 13, further comprising instructions to assist in critical situations being stored on the computer processor and provided on demand to one of the patient and the caregiver.

15. The system of claim 13, wherein the patient or caregiver provides goals and the algorithm tracks progress of the goals.

16. The system of claim 15, wherein the algorithm determines that a goal has been met and the processor reports the met goal to one of the patient and the caregiver.

17. The system of claim 13, further comprising an online portal accessible by a mobile device and configured to send/receive real-time communication and alerts to one of the patient and the caregiver.

18. The system of claim 13, further comprising a connector for operatively connecting to a wearable thermometer, motion detector, pulse oximeter, and/or other sensors to monitor vital signs of the patient, the vital signs being at least part of the personal data.

19. The system of claim 18, further comprising a report generation feature configured to report vital signs of the patient to the caregiver on demand.

20. The system of claim 13, wherein the decision tree algorithm uses machine learning and algorithms to identify a critical glucose data for the patient and instructs an intervention device to automatically treat the patient based on the identified critical glucose data.
